(19) United States
(12) Patent Application Publication
(10) Pub. No.: US 2025/0011304 A1
(43) Pub. Date: Jan. 9, 2025

(54) CRYSTALLINE FORM OF N-(2-CHLORO-3-((5-CHLORO-3-METHYL-4-OXO-3,4-DIHYDROQUINAZOLIN-6-YL)AMINO)-4-FLUOROPHENYL)-3-FLUOROAZETIDINE-1-SULFONAMIDE

(71) Applicant: Array BioPharma Inc., Boulder, CO (US)

(72) Inventor: Connor James COWDREY, Longmont, CO (US)

(73) Assignee: Array BioPharma Inc., Boulder, CO (US)

(21) Appl. No.: 18/709,091

(22) PCT Filed: Dec. 2, 2022
(86) PCT No.: PCT/IB2022/061716

§ 371 Date: May 10, 2024

Related U.S. Application Data
(60) Provisional application No. 63/393,043, filed on Jul. 28, 2022.
(60) Provisional application No. 63/287,127, filed on Dec. 8, 2021.

(51) Int. Cl. C07D 403/12; A61K 9/20; A61K 31/4184; A61K 31/517; A61K 45/06

(52) U.S. Cl. CPC C07D 403/12; A61K 9/2009; A61K 9/2013; A61K 9/2027; A61K 9/2054; A61K 31/4184; A61K 31/517; A61K 45/06

(57) ABSTRACT

This invention relates to a crystalline form of N-(2-chloro-3-((5-chloro-3-methyl-4-oxo-3,4-dihydroquinazolin-6-yl)amino)-4-fluorophenyl)-3-fluoroazetidine-1-sulfonamide, pharmaceutical compositions comprising said crystalline form, and methods of using said crystalline form in the treatment of BRAF-associated diseases and disorders, such as BRAF-associated tumors.

### FIELD OF THE INVENTION

This invention relates to a crystalline anhydrous form of N-(2-chloro-3-((5-chloro-3-methyl-4-oxo-3,4-dihydroquinazolin-6-yl)amino)-4-fluorophenyl)-3-fluoroazetidine-1-sulfonamide, pharmaceutical compositions comprising said crystalline form, and to methods of using said crystalline form in the treatment of BRAF-associated diseases and disorders, including BRAF-associated tumors.

### BACKGROUND OF THE INVENTION

BRAF protein, a member of the RAF family of serine/threonine kinases, participates in the cascade of the Ras-Raf-MEK-extracellular signal-regulated kinase (ERK) pathway or mitogen-activated protein kinase (MAPK)/ERK signaling pathway that affects cell division and differentiation. Mutations in the BRAF gene can lead to uncontrolled growth and subsequent tumor formation. Over 100 unique mutations in the BRAF gene have been identified in cancer (Cerami, E., et al., Cancer Discov. 2012, 2, 401-404). These mutations lead to ERK activation via different functional mechanisms, and have been grouped into three classes, two of which are referred to as Class I and Class II mutations, based on their dependence on dimerization and on activation by RAS for activity; these properties determine their sensitivity to RAF inhibitors (Yao, A., et al., Cancer Cell 2015, 28, 370-383).

Activating Class I BRAF mutations such as V600E and/or V600K have been found in human cancers such as melanoma, colorectal cancer, thyroid cancer, non-small cell lung cancer, ovarian cancer, renal cell carcinoma and metastatic cancers thereof, and primary brain tumors. Class I mutations such as BRAF V600 mutants signal as RAS independent active monomers.

Class II BRAF mutations include non-V600 mutations, which activate MEK through dimerization but without a requirement for RAS (Yao, A., et al., Cancer Cell 2015, 28, 370-383). These Class II mutations undergo constitutive, RAS-independent dimerization, leading to increased ERK activation with low RAS activity due to negative feedback. Common Class II point mutations include G469A/V/R, K601E/N/T, and L597Q/V. Non-V600 mutants are resistant to Class I BRAF inhibitors such as vemurafenib. Non-V600 BRAF mutants have also been found in many cancers and are more prevalent than V600 mutations in certain tumor types. Non-V600 BRAF mutations are found in 5-16% of melanomas, as well as a variety of other tumor types (Siroy A E, et al., J Invest Dermatol. 2015; 135:508-515; Dahlman K B, et al. Cancer Discov. 2012; 2:791-797). Approximately 50-80% of BRAF mutations in non-small cell lung cancer and 22-30% in colorectal cancer encode for non-V600 mutations (Jones J C, et al. J Clin Oncol. 2017; 35:2624-2630; Paik P K, et al. J Clin Oncol. 2011; 29:2046-2051). Class II BRAF mutations such as G469A, G469R, G469V, K601E, K601N, K601T, L597Q and L597V have been identified in gliomas (Schreck, K. C. et al., Cancers (2019) 11:1262) and other tumors such as breast cancer, small cell lung cancer, pancreatic cancer, thyroid cancer, prostate cancer, adenoid cystic carcinoma, appendiceal cancer, small intestine cancer, head and neck squamous cell carcinoma and angiosarcoma (Sullivan, R. J., Cancer Discov Feb. 1 2018 (8) (2) 184-195). Class II BRAF mutations have also been identified in metastatic cancers (Dagogo-Jack, I., Clin Cancer Res. September 2018; Schirripa, M., Clin Cancer Res., May 2019; Menzer, C., J. Clin Oncol 2019, 37(33):3142-3151).

Additionally, BRAF in-frame deletions can function as Class II mutations. For example, acquired resistance has been observed in patients treated with BRAF V600 inhibitors. Mechanisms of acquired resistance include alternated splicing. Splice variants of BRAF encode an active kinase but lack an intact RAS binding domain. Cells resistant to vemurafenib have been found to express variant forms of BRAF V600E that lack exons that encompass the RAS-binding domain, specifically, lacking exons 4-10, exons 4-8, exons 2-8 or exons 2-10 (Poulikakos, P. I, et al., Nature, 480(7377):387-390.

Currently, no effective targeted treatments are available for patients harboring non-V600 BRAF alterations or BRAF inhibitor resistance mutations. Therefore, there remains a need for a treatment for patients harboring non-V600 BRAF alterations or BRAF inhibitor resistance mutations.

Furthermore, although certain inhibitors of BRAF V600 mutations produce excellent extracranial responses, a cancer may still develop brain metastases during, or subsequent to, therapy with BRAF inhibitors (Oliva I. C. G, et al., Annals of Oncology, 29: 1509-1520 (2018)). An estimated 20% of all subjects with cancer will develop brain metastases, with the majority of brain metastases occurring in those with melanoma, colorectal cancer, lung cancer, and renal cell carcinoma (Achrol A. S., et al., Nature Reviews (2019), 5:5, pp 1-26), although these are not the only type of cancer could spread to the brain. Development of brain metastases remains a substantial contributor to overall cancer mortality in subjects with advance-stage cancer because prognosis remains poor despite multimodal treatments and advances in systemic therapies, which includes combinations of surgery, radiotherapy, chemotherapy, immunotherapy, and/or targeted therapies.

BRAF has also been identified as a potential target for treating primary brain tumors. The prevalence of the BRAF-V600E mutation in primary brain tumors has been reported by Schindler et al. (Acta Neuropathol 121(3):397-405, 2011) from the analysis of 1,320 central nervous system (CNS) tumors and by Behling et al. (Diagn Pathol 11(1):55, 2016), who analyzed 969 CNS tumors in pediatric and adult populations. These studies, in combination with others, report the presence of BRAF-V600E mutations in various cancers, including papillary craniopharyngiomas, pleomorphic xanthoastrocytomas (PXAs), gangliogliomas, astroblastomas, and others (Behling et al., Diagn Pathol 11(1):55, 2016; Brastianos et al., Nat Genet 46(2):161-165, 2014; Dougherty et al., Neuro Oncol 12(7):621-630, 2010; Lehman et al., Neuro Oncol 19(1):31-42, 2017; Mordechai et al., Pediatr Hematol Oncol 32(3):207-211, 2015; Myung et al., Transl Oncol 5(6):430-436, 2012; Schindler et al., Acta Neuropathol 121(3):397-405, 2011).

Cancers, including metastatic cancers, having BRAF-fusion proteins have also been described (J. S. Ross, et al., Int. J. Cancer: 138, 881-890 (2016)).

Blood-brain interfaces comprise the cerebral microvessel endothelium forming the blood-brain barrier (BBB) and the epithelium of the choroid plexuses forming the blood-CSF barrier (BCSFB). The blood brain barrier (BBB) is a highly selective physical, transport and metabolic barrier that divides the CNS from the blood. The BBB may prevent certain drugs from entering brain tissue and is a limiting factor in the delivery of many peripherally-administered agents to the CNS. Many drugs commonly used to treat cancer are not able to cross the BBB. This means the drugs are not able to penetrate the brain, and therefore cannot effectively kill cancer cells in the brain. Current treatments for subjects with brain tumors include surgical resection, radiotherapy, and/or chemotherapy with agents such as temozolomide and/or bevacizumab. However, treatment of brain cancers by surgery is not always possible or desirable, for example, the tumor may be inaccessible, or the subject may be incapable of withstanding the trauma of neurosurgery. In addition, radiotherapy and treatment with cytotoxic agents are known to have undesirable side effects. For example, there is increasing evidence that the use of temozolomide may itself induce mutations and worsen prognosis in a significant fraction of subjects (B. E. Johnson et al., Science 343: 189-193 (2014)), and bevacizumab labeling has boxed warnings for gastrointestinal perforation, surgery and wound healing complications, and hemorrhage.

Kinase inhibitors are useful for treating many peripheral cancers. However, due to their structural characteristics, many kinase inhibitors including certain BRAF inhibitors (e.g., vemurafenib and dabrafenib) are substrates of active transporters such as P-glycoproteins (P-gp) or breast cancer resistance protein (BCRP). For example, dabrafenib has been reported to have an MDR1 efflux ratio of 11.4, a BCRP efflux ratio of 21.0, and a total brain-to-plasma ratio of 0.023; a free brain-to-plasma ratio was not reported (Mittapalli, R K, et al., J Pharmacol. Exp Ther 344:655-664, March 2013), and vemurafenib has been reported to have an MDR1 efflux ratio of 83, a BCRP efflux ratio of 495, and a total brain-to-plasma ratio of 0.004; a free brain-to-plasma ratio was not reported (Mittapalli, R K. et al., J Pharmacol. Exp Ther 342:33-40 (March 2012). Given that both P-gp and BCRP are expressed in the endothelial cells lining the blood brain capillaries, the activity of both P-gp and BCRP in the BBB play a critical role in preventing the distribution of most kinase inhibitors to the brain parenchyma. Therefore, kinase inhibitors are not generally suitable to be used for the treatment of tumors or cancers in the brain, which is protected by the BBB.

The compound N-(2-chloro-3-((5-chloro-3-methyl-4-oxo-3,4-dihydroquinazolin-6-yl)amino)-4-fluorophenyl)-3-fluoroazetidine-1-sulfonamide is a potent inhibitor of both BRAF Class I and Class II mutations and may be useful for the treatment of BRAF-associated diseases and disorders, including BRAF-associated tumors.

In addition, N-(2-chloro-3-((5-chloro-3-methyl-4-oxo-3,4-dihydroquinazolin-6-yl)amino)-4-fluorophenyl)-3-fluoroazetidine-1-sulfonamide is not a substrate of the active transporters P-glycoproteins (P-gp) or breast cancer resistance protein (BCRP) and therefore may be useful for the treatment of malignant and benign BRAF-associated tumors of the CNS and malignant extracranial BRAF-associated tumors.

The free base of N-(2-chloro-3-((5-chloro-3-methyl-4-oxo-3,4-dihydroquinazolin-6-yl)amino)-4-fluorophenyl)-3-fluoroazetidine-1-sulfonamide (hereinafter “Compound 1”) has the structure shown below:

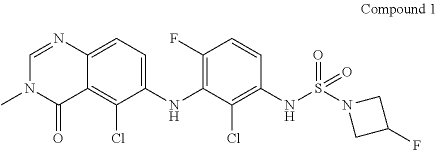 [SMILES: Cn1cnc2ccc(Nc3c(F)ccc(NS(=O)(=O)N4CC(F)C4)c3Cl)c(Cl)c2c1=O]

It is desirable to identify a form of Compound 1 having good physical-chemical properties such as good physical stability and non-hygroscopicity that can provide better quality control of drug product manufacturing process and drug product compositions.

Preparation of the free base of Compound 1 is disclosed in Example 126 of International Patent Application No. PCT/IB2021/054919 filed Jun. 4, 2021, the contents of which are incorporated herein by reference in its entirety.

### BRIEF SUMMARY OF THE INVENTION

Each of the embodiments described below can be combined with any other embodiment described herein not inconsistent with the embodiment with which it is combined.

In one aspect, the invention provides crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide (hereinafter “crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1” or “crystalline anhydrous Compound 1, Form 1”).

In another aspect, the invention provides a pharmaceutical composition comprising crystalline anhydrous Compound 1, Form 1, and one or more pharmaceutically acceptable excipients.

In another aspect, the invention provides a method of treating a BRAF-associated disease or disorder, comprising administering to a subject in need thereof a therapeutically effective amount of crystalline anhydrous Compound 1, Form 1.

In another aspect, the invention provides a method of treating a BRAF-associated disease or disorder, comprising administering to a subject in need thereof a therapeutically effective amount of a pharmaceutical composition comprising crystalline anhydrous Compound 1, Form 1.

In another aspect, the invention provides use of crystalline anhydrous Compound 1, Form 1 for the manufacture of a medicament for the treatment of a BRAF-associated disease or disorder.

In another aspect, the invention provides crystalline anhydrous Compound 1, Form 1 for use as a medicament.

In another aspect, the invention provides crystalline anhydrous Compound 1, Form 1 for use in the treatment of a BRAF-associated disease or disorder.

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

### BRIEF DESCRIPTION OF THE DRAWINGS

FIG. 1 depicts a powder X-ray diffraction pattern of crystalline anhydrous Compound 1, Form 1.

FIG. 2 depicts a solid state ¹⁹F solid state NMR spectrum of crystalline anhydrous Compound 1, Form 1, wherein the peaks (chemical shift in ppm) marked by hashes (#) indicate spinning sidebands.

FIG. 3. depicts a solid state ¹³C solid state NMR spectrum of crystalline anhydrous Compound 1, Form 1, wherein the peaks (chemical shift in ppm) marked by hashes (#) indicate spinning sidebands.

FIG. 4. depicts a Raman spectrum of crystalline anhydrous Compound 1, Form 1 (Raman shift in cm⁻¹).

FIG. 5. depicts a powder X-ray diffraction pattern of amorphous Compound 1, Form 2.

### DETAILED DESCRIPTION OF THE INVENTION

The present invention may be understood more readily by reference to the following detailed description of the invention and the Examples included herein. It is to be understood that this invention is not limited to specific synthetic methods of making that may of course vary. It is to be also understood that the terminology used herein is for the purpose of describing specific embodiments only and is not intended to be limiting.

The present invention is directed to crystalline anhydrous Compound 1, Form 1. The present invention is also directed to pharmaceutical compositions comprising such crystalline form. The present invention is also directed to methods of using such crystalline form in the treatment of BRAF-associated diseases or disorders.

The term “crystalline” as used herein, means having a regularly repeating arrangement of molecules or external face planes. Crystalline forms may differ with respect to thermodynamic stability, physical parameters, X-ray structure and preparation processes.

The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’).

There are a number of analytical methods one of ordinary skill in the art in solid-state chemistry can use to analyze solid forms. Powder X-ray diffraction may also be suitable for quantifying the amount of a crystalline solid form (or forms) in a mixture. In powder X-ray diffraction, X-rays are directed onto a crystalline powder and the intensity of the diffracted X-rays is measured as a function of the angle between the X-ray source and the beam diffracted by the sample. The intensity of these diffracted X-rays can be plotted on a graph as peaks with the x-axis being the angle (this is known as the “2-theta” angle) between the X-ray source and the diffracted X-rays and with the y-axis being the intensity of the diffracted X-rays. This graph is called a powder X-ray diffraction pattern or powder pattern. Different crystalline solid forms exhibit different powder patterns because the location of the peaks on the x-axis is a property of the solid-state structure of the crystal.

Due to differences in instruments, samples, and sample preparation, peak values are sometimes reported with the modifier for the peak values. This is common practice in the solid-state chemical arts because of the variation inherent in peak values. One of ordinary skill in the art will appreciate that a typical precision of the 2-theta x-axis value of a peak in a powder pattern is on the order of plus or minus 0.2° 2-theta (±0.2° 2θ). Thus, for example, a diffraction peak that appears at “8.3° 2-theta” means that the peak could be between 8.1° 2-theta and 8.5° 2-theta when measured on most X-ray diffractometers under most conditions. Further, one skilled in the art will appreciate that the relative peak intensities will show inter-apparatus variability as well as variability due to degree of crystallinity, preferred orientation, prepared sample surface, and other factors known to those skilled in the art and should be taken as qualitative measures only.

Powder X-ray diffraction is just one of several analytical techniques one may use to characterize and/or identify crystalline solid forms. Spectroscopic techniques such as Raman (including microscopic Raman), infrared, and solid state NMR spectroscopies may be used to characterize and/or identify crystalline solid forms. These techniques may also be used to quantify the amount of one or more crystalline solid forms in a mixture. A typical variability for a wave number associated with an FT-Raman and FT-Infrared measurement is on the order of plus or minus (±) 2 cm⁻¹. A typical variability for a chemical shift associated with a ¹³C or ¹⁹F NMR is of the order of plus or minus (±) 0.2 ppm for crystalline material. A typical variability for a value associated with a differential scanning calorimetry onset temperature is of the order of plus or minus (±) 5° C.

As used herein, the term “essentially the same” means that variability typical for a particular method is taken into account. For example, with reference to powder X-ray diffraction peak positions, the term “essentially the same” means that typical variability in peak position and intensity are taken into account. One skilled in the art will appreciate that the peak positions (2-theta) will show some variability, typically as much as ±0.2° 2θ. Further, one skilled in the art will appreciate that relative peak intensities will show inter-apparatus variability, as well as variability due to the degree of crystallinity, preferred orientation, prepared sample surface, and other factors known to those skilled in the art and should be taken as qualitative measures only. A typical variability for a chemical shift associated with a ¹³C or ¹⁹F NMR is on the order of ±0.2 ppm for crystalline material.

As used herein, the singular form “a”, “an”, and “the” include plural references unless indicated otherwise.

Unless otherwise defined herein, the term “about” means having a value falling within an accepted standard of error of the mean, when considered by one of ordinary skill in the art. In one embodiment the term about means plus or minus 10%.

In some embodiments, crystalline anhydrous Compound 1, Form 1 may be substantially pure. As used herein, the term “substantially pure” means with reference to crystalline anhydrous Compound 1, Form 1, that the crystalline form includes less than 10%, preferably less than 5%, preferably less than 3%, preferably less than 1% by weight of any other physical form of Compound 1 on a weight basis. In one embodiment the term “substantially pure” means a crystalline anhydrous Compound 1, Form 1 contains less than about 10% of any other physical form of Compound 1 on a weight basis. In one embodiment the term “substantially pure” means crystalline anhydrous Compound 1, Form 1 contains less than about 5% of any other physical form of Compound 1 on a weight basis. In one embodiment the term “substantially pure” means crystalline anhydrous Compound 1, Form 1 contains less than about 3% of any other physical form of Compound 1 on a weight basis. In one embodiment the term “substantially pure” means crystalline anhydrous Compound 1, Form 1 contains less than about 1% of any other physical form of Compound 1 on a weight basis.

The term “anhydrous” as used herein, refers to a crystalline form without any solvent or water molecules in the crystal lattice.

### Crystalline Compound 1

In one aspect, provided herein is crystalline anhydrous Compound 1, Form 1.

As described herein, crystalline anhydrous Compound 1, Form 1 can be characterized by any of the following methods: (1) powder X-ray diffraction (PXRD) (2-theta); (2) ¹⁹F solid state NMR spectroscopy (ppm); (3) ¹³C solid state NMR spectroscopy; (4) Raman spectroscopy; or a combination any two or more of methods (1), (2), (3) and (4).

In each of the aspects and embodiments herein that are characterized by PXRD, the PXRD peaks were collected using CuKα̅ radiation at 1.5418λ.

Crystalline anhydrous Compound 1, Form 1 may be further characterized by additional techniques, such as differential scanning calorimetry (DSC), Fourier transform infrared spectroscopy (FTIR), thermogravimetric analysis (TGA), or differential thermal analysis (DTA).

In one embodiment, crystalline anhydrous Compound 1, Form 1 is characterized by its powder X-ray diffraction (PXRD) pattern.

Table 1 provides a full PXRD peak list for crystalline anhydrous Compound 1, Form 1 in degrees 2-theta (±0.2 degrees 2-theta).

TABLE 1

| Angle | Rel. Intensity |
|---|---|
| (°2-Theta) | (%) |
| 8.3 | 100 |
| 11.5 | 65.4 |
| 16.1 | 17 |
| 16.4 | 24.7 |
| 16.8 | 4.5 |
| 18.1 | 8 |
| 22.5 | 5.6 |
| 22.9 | 24.7 |
| 23.6 | 25.4 |
| 23.7 | 18.8 |
| 24.1 | 6.8 |
| 26.3 | 15 |
| 26.5 | 4.6 |
| 26.9 | 9.2 |
| 28.1 | 3.6 |
| 29.3 | 6.7 |
| 30.2 | 5.3 |
| 31.3 | 4.5 |
| 31.4 | 5.6 |
| 32.3 | 3.1 |

In one embodiment, the invention provides crystalline anhydrous Compound 1, Form 1 having a PXRD pattern comprising characterizing peaks, in terms of 2-theta, at 8.3, 11.5 and 16.1 degrees 2-theta (±0.2 degrees 2-theta). In another embodiment, Form 1 having a PXRD pattern comprising characterizing peaks, in terms of 2-theta, at 8.3, 11.5, 16.1, 22.9, and 23.6 degrees 2-theta (±0.2 degrees 2-theta).

In one embodiment, the invention provides crystalline anhydrous Compound 1, Form 1 having a PXRD pattern comprising a PXRD peak listing essentially the same as in Table 1 in degrees 2-theta (i 0.2 degrees 2-theta).

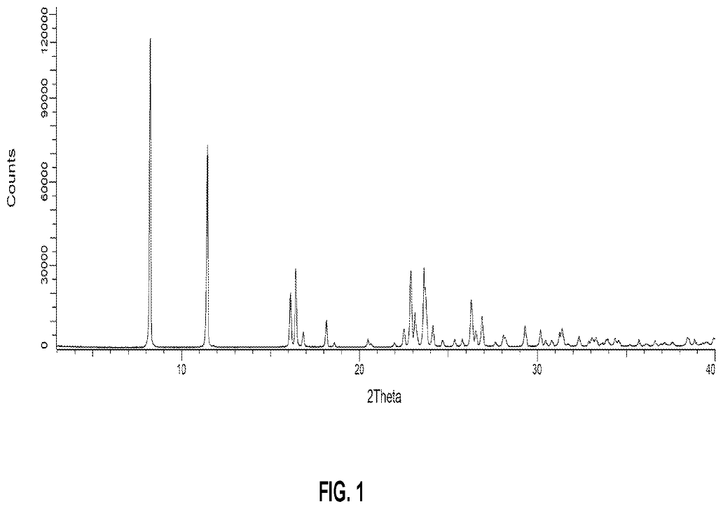

In one embodiment, the invention provides crystalline anhydrous Compound 1, Form 1 having a PXRD pattern comprising peaks at 2-theta values essentially the same as shown in FIG. 1.

In one embodiment of the invention, crystalline anhydrous Compound 1, Form 1 is characterized by its ¹⁹F solid state NMR spectrum.

In one embodiment, provided herein is crystalline anhydrous Compound 1, Form 1 having a ¹⁹F solid state NMR spectrum comprising at least one resonance value (ppm) selected from the values shown in Table 2 (ppm)±0.2 ppm.

TABLE 2

| ¹⁹F Chemical Shift (ppm) | Relative Intensity (%) |
|---|---|
| −188.1 | 100 |
| −115.8 | 56 |

In one embodiment, provided herein is crystalline anhydrous Compound 1, Form 1 having a ¹⁹F solid state NMR spectrum comprising a resonance value of −188.1 ppm±0.2 ppm.

In one embodiment, provided herein is crystalline anhydrous Compound 1, Form 1 having a ¹⁹F solid state NMR spectrum comprising a resonance value of −115.8 ppm±0.2 ppm.

In one embodiment, provided herein is crystalline anhydrous Compound 1, Form 1 having a ¹⁹F solid state NMR spectrum comprising resonance values of −188.1 and −115.8 ppm±0.2 ppm.

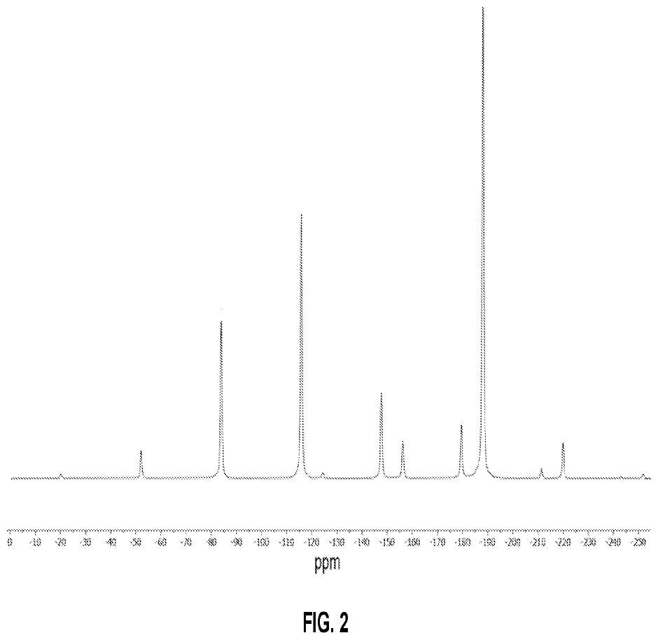

In one embodiment, provided herein is crystalline anhydrous Compound 1, Form 1 having a ¹⁹F solid state NMR spectrum comprising resonance (ppm) values essentially the same as shown in FIG. 2, in which the peaks marked by hashes (#) are spinning side bands.

In one embodiment of the invention, crystalline anhydrous Compound 1, Form 1 is characterized by its ¹³C solid state NMR spectrum.

Table 3A provides a full ¹³C solid state NMR peak list for crystalline anhydrous Compound 1, Form 1 in ppm±0.2 ppm.

TABLE 3A

| ¹³C Chemical Shift | Relative Intensity |
|---|---|
| (ppm) | (%) |
| 159.1 | 27 |
| 157.5 | 14 |
| 155.4 | 21 |
| 148.1 | 63 |
| 143.3 | 37 |
| 140.0 | 40 |
| 130.6 | 45 |
| 127.3 | 77 |
| 126.7 | 78 |
| 125.2 | 36 |
| 119.1 | 60 |
| 115.1 | 10 |
| 112.7 | 59 |
| 87.1 | 30 |
| 85.6 | 22 |
| 57.5 | 100 |
| 35.8 | 98 |

In one embodiment, provided herein is crystalline anhydrous Compound 1, Form 1 characterized by a ¹³C solid state NMR spectrum comprising one or more resonance values shown in Table 3B (ppm)±0.2 ppm.

TABLE 3B
Characteristic ¹³C solid state NMR peak list for crystalline
anhydrous Compound 1, Form 1 (±0.2 ppm)

| ¹³C Chemical Shift (ppm) | Relative Intensity (%) |
|---|---|
| 35.8 | 98 |
| 57.5 | 100 |
| 130.6 | 45 |
| 148.1 | 63 |

In one embodiment, provided herein is crystalline anhydrous Compound 1, Form 1 having a ¹³C solid state NMR spectrum comprising resonance values at 35.8 and 57.5 ppm±0.2 ppm.

In one embodiment, provided herein is crystalline anhydrous Compound 1, Form 1 having a ¹³C solid state NMR spectrum comprising resonance values at 57.5 and 148.1 ppm±0.2 ppm.

In one embodiment, provided herein is crystalline anhydrous Compound 1, Form 1 having a ¹³C solid state NMR spectrum comprising resonance values at 57.5 and 130.6 ppm±0.2 ppm.

In one embodiment, provided herein is crystalline anhydrous Compound 1, Form 1 having a ¹³C solid state NMR spectrum comprising resonance values at 35.8, 57.5 and 148.1 ppm±0.2 ppm.

In one embodiment, provided herein is crystalline anhydrous Compound 1, Form 1 having a ¹³C solid state NMR spectrum comprising resonance values at 57.5, 130.6 and 148.1 ppm±0.2 ppm.

In one embodiment, provided herein is crystalline anhydrous Compound 1, Form 1 having a ¹³C solid state NMR spectrum comprising resonance values at 35.8, 57.5, 130.6 and 148.1 ppm±0.2 ppm.

In one embodiment, provided herein is crystalline anhydrous Compound 1, Form 1 having a ¹³C solid state NMR spectrum comprising the resonance values (ppm) shown in Table 3A±0.2 ppm.

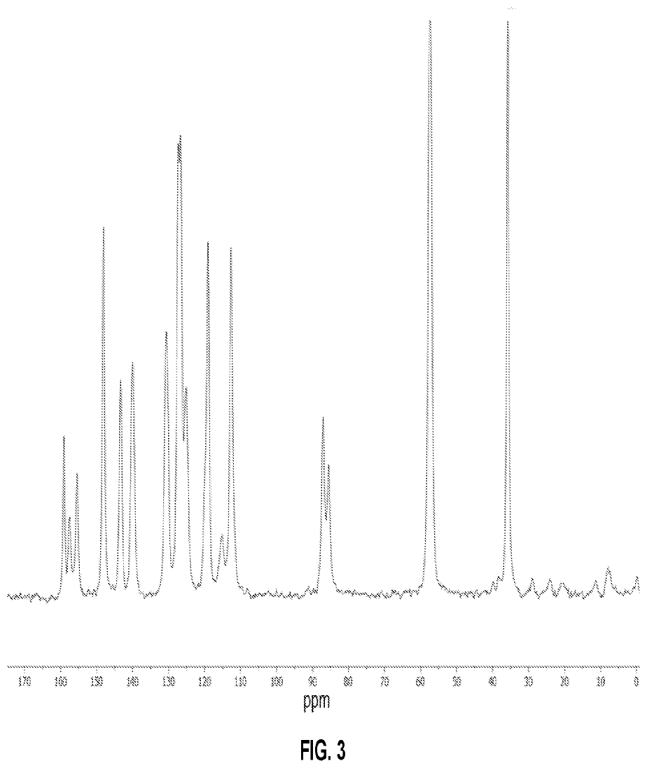

In one embodiment, provided herein is crystalline anhydrous Compound 1, Form 1 having a ¹³C solid state NMR spectrum comprising resonance (ppm) values essentially the same as shown in FIG. 3±0.2 ppm, in which the peaks marked by hashes (#) are spinning side bands.

In one embodiment of the invention, crystalline anhydrous Compound 1, Form 1 is characterized by its Raman spectrum.

Table 4A provides a full Raman peak list for crystalline anhydrous Compound 1, Form 1.

TABLE 4A

| Position (cm⁻¹) | Normalized Intensity |
|---|---|
| 216 | 0.10 |
| 237 | 0.08 |
| 259 | 0.07 |
| 280 | 0.11 |
| 306 | 0.08 |
| 339 | 0.15 |
| 370 | 0.14 |
| 404 | 0.17 |
| 424 | 0.21 |
| 460 | 0.17 |
| 477 | 0.10 |
| 487 | 0.10 |
| 514 | 0.11 |
| 528 | 0.08 |
| 546 | 0.15 |
| 560 | 0.13 |
| 592 | 0.07 |
| 615 | 0.09 |
| 632 | 0.79 |
| 644 | 0.11 |
| 665 | 0.12 |
| 702 | 0.08 |
| 712 | 0.10 |
| 745 | 0.08 |
| 797 | 0.10 |
| 814 | 0.25 |
| 826 | 0.10 |
| 852 | 0.09 |
| 887 | 0.14 |
| 935 | 0.10 |
| 1017 | 0.09 |
| 1057 | 0.19 |
| 1076 | 0.11 |
| 1134 | 0.13 |
| 1147 | 0.11 |
| 1173 | 0.14 |
| 1192 | 0.09 |
| 1232 | 0.25 |
| 1256 | 0.17 |
| 1308 | 0.49 |
| 1358 | 0.13 |
| 1373 | 0.10 |
| 1398 | 0.24 |
| 1409 | 0.19 |
| 1433 | 0.30 |
| 1447 | 0.33 |
| 1477 | 0.11 |
| 1493 | 0.10 |
| 1515 | 0.13 |
| 1548 | 0.50 |
| 1608 | 1.00 |
| 1673 | 0.17 |
| 1863 | 0.07 |
| 2863 | 0.05 |
| 2901 | 0.05 |
| 2951 | 0.12 |
| 2989 | 0.10 |
| 3053 | 0.13 |
| 3078 | 0.10 |
| 3099 | 0.09 |
| 3378 | 0.12 |

In one embodiment, provided herein is crystalline anhydrous Compound 1, Form 1 having a Raman spectrum comprising one or more wavenumber (cm⁻¹) values selected from Table 4B±2 cm⁻¹.

TABLE 4B

| Position (cm⁻¹) | Normalized Intensity |
|---|---|
| 1308 | 0.49 |
| 1433 | 0.30 |
| 1447 | 0.33 |
| 1548 | 0.50 |
| 1608 | 1.00 |

In one embodiment, provided herein is crystalline anhydrous Compound 1, Form 1 having a Raman spectrum comprising wavenumber (cm⁻¹) values at 1548 and 1608 cm⁻¹±2 cm⁻¹.

In one embodiment, provided herein is crystalline anhydrous Compound 1, Form 1 having a Raman spectrum comprising wavenumber (cm⁻¹) values at 1308 and 1608 cm⁻¹±2 cm⁻¹.

In one embodiment, provided herein is crystalline anhydrous Compound 1, Form 1 having a Raman spectrum comprising wavenumber (cm⁻¹) values at 1447 and 1608 cm⁻¹±2 cm⁻¹.

In one embodiment, provided herein is crystalline anhydrous Compound 1, Form 1 having a Raman spectrum comprising wavenumber (cm⁻¹) values at 1433 and 1608 cm⁻¹±2 cm⁻¹.

In one embodiment, provided herein is crystalline anhydrous Compound 1, Form 1 having a Raman spectrum comprising wavenumber (cm⁻¹) values at 1308, 1548 and 1608 cm⁻¹±2 cm⁻¹.

In one embodiment, provided herein is crystalline anhydrous Compound 1, Form 1 having a Raman spectrum comprising wavenumber (cm⁻¹) values at 1308, 1447, 1548 and 1608 cm⁻¹±2 cm⁻¹.

In one embodiment, provided herein is crystalline anhydrous Compound 1, Form 1 having a Raman spectrum comprising wavenumber (cm⁻¹) values at 1308, 1433, 1447, 1548 and 1608 cm⁻¹±2 cm⁻¹.

In one embodiment, provided herein is crystalline anhydrous Compound 1, Form 1 having a Raman spectrum comprising the wavenumber (cm⁻¹) values shown in Table 4A±2 cm⁻¹.

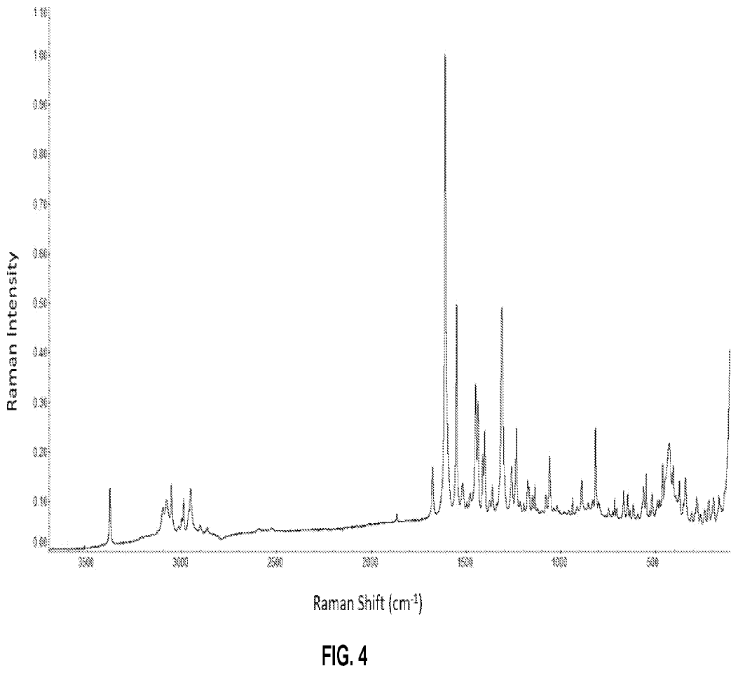

In one embodiment, provided herein is crystalline anhydrous Compound 1, Form 1 having a Raman spectrum essentially the same as shown in FIG. 4.

In one embodiment, crystalline anhydrous Compound 1, Form 1 is characterized by its powder X-ray diffraction (PXRD) pattern and its ¹⁹F solid state NMR spectrum.

In one embodiment, provided herein is crystalline anhydrous Compound 1, Form 1 having: (a) a PXRD pattern comprising peaks at 2-theta values of 8.3, 11.5 and 16.1 degrees 2-theta (±0.2 degrees 2-theta) and (b) a ¹⁹F solid state NMR spectrum comprising resonance values at −188.1 and −115.8 ppm±0.2 ppm.

In one embodiment, provided herein is crystalline anhydrous Compound 1, Form 1 having: (a) a PXRD pattern comprising peaks at 2-theta values of 8.3, 11.5 and 16.1 degrees 2-theta (±0.2 degrees 2-theta) and (b) a ¹⁹F solid state NMR spectrum comprising a resonance value at −188.1 ppm±0.2 ppm.

In one embodiment, provided herein is crystalline anhydrous Compound 1, Form 1 having: (a) a PXRD pattern comprising peaks at 2-theta values of 8.3, 11.5 and 16.1 degrees 2-theta (±0.2 degrees 2-theta) and (b) a ¹⁹F solid state NMR spectrum comprising a resonance value at −115.8 ppm±0.2 ppm.

In one embodiment, crystalline anhydrous Compound 1, Form 1 is characterized by its powder X-ray diffraction (PXRD) pattern and its ¹³C solid state NMR spectrum.

In one embodiment, provided herein is crystalline anhydrous Compound 1, Form 1 having: (a) a PXRD pattern comprising peaks at 2-theta values of 8.3, 11.5 and 16.1 degrees 2-theta (±0.2 degrees 2-theta) and (b) a ¹³C solid state NMR comprising resonance values at 35.8, 57.5, 130.6 and 148.1 ppm±0.2 ppm.

In one embodiment, crystalline anhydrous Compound 1, Form 1 is characterized by its powder X-ray diffraction (PXRD) pattern and its Raman spectrum.

In one embodiment, provided herein is crystalline anhydrous Compound 1, Form 1 having: (a) a PXRD pattern comprising peaks at 2-theta values of 8.3, 11.5 and 16.1 degrees 2-theta (±0.2 degrees 2-theta) and (b) a Raman spectrum comprising wavenumber (cm⁻¹) values at 1308, 1433, 1447, 1548 and 1608 cm⁻¹±2 cm⁻¹.

In one embodiment, crystalline anhydrous Compound 1, Form 1 is characterized by its ¹⁹F solid state NMR spectrum and its Raman spectrum.

In one embodiment, provided herein is crystalline anhydrous Compound 1, Form 1 having: (a) a ¹⁹F solid state NMR spectrum comprising resonance values at −188.1 and −115.8 ppm±0.2 ppm, and (b) a Raman spectrum comprising wavenumber (cm⁻¹) values at 1308, 1433, 1447, 1548 and 1608 cm⁻¹±2 cm⁻¹.

In one embodiment, provided herein is crystalline anhydrous Compound 1, Form 1 having: (a) a ¹⁹F solid state NMR spectrum comprising a resonance value at −188.1 ppm±0.2 ppm, and (b) a set of Raman bands at 1308, 1433, 1447, 1548 and 1608 cm⁻¹±2 cm⁻¹.

In one embodiment, provided herein is crystalline anhydrous Compound 1, Form 1 having: (a) a ¹⁹F solid state NMR spectrum comprising a resonance value at −115.8 ppm±0.2 ppm, and (b) a Raman spectrum comprising wavenumber (cm⁻¹) values at 1308, 1433, 1447, 1548 and 1608 cm⁻¹±2 cm⁻¹.

In one embodiment, crystalline anhydrous Compound 1, Form 1 is characterized by its ¹⁹F solid state NMR spectrum and its ¹³C solid state NMR spectrum.

In one embodiment, provided herein is crystalline anhydrous Compound 1, Form 1 having: (a) a ¹⁹F solid state NMR spectrum comprising resonance values at −188.1 and −115.8 ppm±0.2 ppm, and (b) a ¹³C solid state NMR comprising resonance values at 35.8, 57.5, 130.6 and 148.1 ppm±0.2 ppm.

In one embodiment, provided herein is crystalline anhydrous Compound 1, Form 1 having: (a) a ¹⁹F solid state NMR spectrum comprising a resonance value at −115.8 ppm±0.2 ppm, and (b) a ¹³C solid state NMR comprising resonance values at 35.8, 57.5, 130.6 and 148.1 ppm±0.2 ppm.

In one embodiment, provided herein is crystalline anhydrous Compound 1, Form 1 having: a ¹⁹F solid state NMR spectrum comprising a resonance value at −115.8 ppm±0.2 ppm and (b) a ¹³C solid state NMR comprising resonance values at 35.8, 57.5, 130.6 and 148.1 ppm±0.2 ppm.

In one embodiment, crystalline anhydrous Compound 1, Form 1 is characterized by its Raman spectrum and its ¹³C solid state NMR spectrum.

In one embodiment, provided herein is crystalline anhydrous Compound 1, Form 1 having: (a) a Raman spectrum comprising wavenumber (cm¹) values at 1308, 1433, 1447, 1548 and 1608 cm⁻¹±2 cm⁻¹ and (b) a ¹³C solid state NMR comprising resonance values at 35.8, 57.5, 130.6 and 148.1 ppm±0.2 ppm.

In one embodiment, crystalline anhydrous Compound 1, Form 1 is characterized by its powder X-ray diffraction (PXRD) pattern, its ¹⁹F solid state NMR spectrum, and its ¹³C solid state NMR spectrum.

In one embodiment, provided herein is crystalline anhydrous Compound 1, Form 1 having (a) a PXRD pattern comprising peaks at 2-theta values of 8.3, 11.5 and 16.1 degrees 2-theta (±0.2 degrees 2-theta), (b) a ¹⁹F solid state NMR spectrum comprising resonance values at −188.1 and −115.8 ppm±0.2 ppm, and (c) a ¹³C solid state NMR comprising resonance values at 35.8, 57.5, 130.6 and 148.1 ppm±0.2 ppm.

In one embodiment, provided herein is crystalline anhydrous Compound 1, Form 1 having (a) a PXRD pattern comprising peaks at 2-theta values of 8.3, 11.5 and 16.1 degrees 2-theta (±0.2 degrees 2-theta), (b) a ¹⁹F solid state NMR spectrum comprising a resonance value at −188.1 ppm±0.2 ppm, and (c) a ¹³C solid state NMR comprising resonance values at 35.8, 57.5, 130.6 and 148.1 ppm±0.2 ppm.

In one embodiment, provided herein is crystalline anhydrous Compound 1, Form 1 having (a) a PXRD pattern comprising peaks at 2-theta values of 8.3, 11.5 and 16.1 degrees 2-theta (±0.2 degrees 2-theta), (b) a ¹⁹F solid state NMR spectrum comprising a resonance value at −115.8 ppm±0.2 ppm, and (c) a ¹³C solid state NMR comprising resonance values at 35.8, 57.5, 130.6 and 148.1 ppm±0.2 ppm.

In one embodiment, crystalline anhydrous Compound 1, Form 1 is characterized by its powder X-ray diffraction (PXRD) pattern, its ¹⁹F solid state NMR spectrum, and its Raman spectrum.

In one embodiment, provided herein is crystalline anhydrous Compound 1, Form 1 having (a) a PXRD pattern comprising peaks at 2-theta values of 8.3, 11.5 and 16.1 degrees 2-theta (±0.2 degrees 2-theta), (b) a ¹⁹F solid state NMR spectrum comprising resonance values at −188.1 and −115.8 ppm±0.2 ppm, and (c) a Raman spectrum comprising wavenumber (cm⁻¹) values at 1308, 1433, 1447, 1548 and 1608 cm⁻¹±2 cm⁻¹.

In one embodiment, provided herein is crystalline anhydrous Compound 1, Form 1 having (a) a PXRD pattern comprising peaks at 2-theta values of 8.3, 11.5 and 16.1 degrees 2-theta (±0.2 degrees 2-theta), (b) a ¹⁹F solid state NMR spectrum comprising a resonance value at −188.1 ppm±0.2 ppm, and (c) a Raman spectrum comprising wavenumber (cm⁻¹) values at 1308, 1433, 1447, 1548 and 1608 cm⁻¹±2 cm⁻¹.

In one embodiment, provided herein is crystalline anhydrous Compound 1, Form 1 having (a) a PXRD pattern comprising peaks at 2-theta values of 8.3, 11.5 and 16.1 degrees 2-theta (±0.2 degrees 2-theta), (b) a ¹⁹F solid state NMR spectrum comprising a resonance value at −115.8 ppm±0.2 ppm, and (c) a Raman spectrum comprising wavenumber (cm⁻¹) values at 1308, 1433, 1447, 1548 and 1608 cm⁻¹±2 cm⁻¹.

In one embodiment, crystalline anhydrous Compound 1, Form 1 is characterized by its ¹⁹F solid state NMR spectrum, its ¹³C solid state NMR spectrum, and its Raman spectrum.

In one embodiment, provided herein is crystalline anhydrous Compound 1, Form 1 having: (a) a ¹⁹F solid state NMR spectrum comprising resonance values at −188.1 and −115.8 ppm±0.2 ppm, (b) a ¹³C solid state NMR comprising resonance values at 35.8, 57.5, 130.6 and 148.1 ppm±0.2 ppm, and (c) a Raman spectrum comprising wavenumber (cm⁻¹) values at 1308, 1433, 1447, 1548 and 1608 cm⁻¹±2 cm⁻¹.

In one embodiment, provided herein is crystalline anhydrous Compound 1, Form 1 having: (a) a ¹⁹F solid state NMR spectrum comprising a resonance value at −188.1 ppm±0.2 ppm, (b) a ¹³C solid state NMR comprising resonance values at 35.8, 57.5, 130.6 and 148.1 ppm±0.2 ppm, and (c) a Raman spectrum comprising wavenumber (cm⁻¹) values at 1308, 1433, 1447, 1548 and 1608 cm⁻¹±2 cm⁻¹.

In one embodiment, provided herein is crystalline anhydrous Compound 1, Form 1 having (a) a ¹⁹F solid state NMR spectrum comprising a resonance value at −115.8 ppm±0.2 ppm, (b) a ¹³C solid state NMR comprising resonance values at 35.8, 57.5, 130.6 and 148.1 ppm±0.2 ppm, and (c) a Raman spectrum comprising wavenumber (cm⁻¹) values at 1308, 1433, 1447, 1548 and 1608 cm⁻¹±2 cm⁻¹.

In one embodiment, crystalline anhydrous Compound 1, Form 1 is characterized by its powder X-ray diffraction (PXRD) pattern, its ¹³C solid state NMR spectrum, and its Raman spectrum.

In one embodiment, provided herein is crystalline anhydrous Compound 1, Form 1 having (a) a PXRD pattern comprising peaks at 2-theta values of 8.3, 11.5 and 16.1 degrees 2-theta (±0.2 degrees 2-theta), (c) a ¹³C solid state NMR comprising resonance values at 35.8, 57.5, 130.6 and 148.1 ppm±0.2 ppm, and (d) a Raman spectrum comprising wavenumber (cm⁻¹) values at 1308, 1433, 1447, 1548 and 1608 cm⁻¹±2 cm⁻¹.

In one embodiment, crystalline anhydrous Compound 1, Form 1 is characterized by its powder X-ray diffraction (PXRD) pattern, its ¹⁹F solid state NMR spectrum, its ¹³C solid state NMR spectrum, and its Raman spectrum.

In one embodiment, provided herein is crystalline anhydrous Compound 1, Form 1 having (a) a PXRD pattern comprising peaks at 2-theta values of 8.3, 11.5 and 16.1 degrees 2-theta (±0.2 degrees 2-theta), (b) a ¹⁹F solid state NMR spectrum comprising resonance values at −188.1 and −115.8 ppm±0.2 ppm, (c) a ¹³C solid state NMR comprising resonance values at 35.8, 57.5, 130.6 and 148.1 ppm±0.2 ppm and (d) a Raman spectrum comprising wavenumber (cm⁻¹) values at 1308, 1433, 1447, 1548 and 1608 cm⁻¹±2 cm⁻¹.

In one embodiment, provided herein is crystalline anhydrous Compound 1, Form 1 having (a) a PXRD pattern comprising peaks at 2-theta values of 8.3, 11.5 and 16.1 degrees 2-theta (±0.2 degrees 2-theta), (b) a ¹⁹F solid state NMR spectrum comprising a resonance value at −188.1 ppm±0.2 ppm, (c) a ¹³C solid state NMR comprising resonance values at 35.8, 57.5, 130.6 and 148.1 ppm±0.2 ppm, and (d) a Raman spectrum comprising wavenumber (cm⁻¹) values at 1308, 1433, 1447, 1548 and 1608 cm⁻¹±2 cm⁻¹.

In one embodiment, provided herein is crystalline anhydrous Compound 1, Form 1 having (a) a PXRD pattern comprising peaks at 2-theta values of 8.3, 11.5 and 16.1 degrees 2-theta (±0.2 degrees 2-theta), (b) a ¹⁹F solid state NMR spectrum comprising a resonance value at −115.8 ppm±0.2 ppm, (c) a ¹³C solid state NMR comprising resonance values at 35.8, 57.5, 130.6 and 148.1 ppm±0.2 ppm and (d) a Raman spectrum comprising wavenumber (cm⁻¹) values at 1308, 1433, 1447, 1548 and 1608 cm⁻¹±2 cm⁻¹.

In one embodiment, crystalline anhydrous Compound 1, Form 1 is substantially pure.

In certain embodiments, crystalline anhydrous Compound 1, Form 1 is greater than 95% substantially pure.

In certain embodiments, crystalline anhydrous Compound 1, Form 1 is greater than 97% substantially pure.

In certain embodiments, crystalline anhydrous Compound 1, Form 1 is greater than 99% substantially pure.

### Pharmaceutical Compositions

In one embodiment, provided herein are pharmaceutical compositions for delivery of crystalline anhydrous Compound 1, Form 1.

In one embodiment, a pharmaceutical composition comprises crystalline anhydrous Compound 1, Form 1 and one or more pharmaceutically acceptable excipients.

The term “excipient” is used herein to describe any ingredient other than crystalline anhydrous Compound 1, Form 1. The choice of excipient will to a large extent depend on factors such as the mode of administration, the effect of the excipient on solubility and stability, and the nature of the dosage form. Said excipient has no pharmaceutical activity on its own.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate (e.g., dibasic calcium phosphate anhydrous), various sugars and types of starch, cellulose derivatives, gelatin, vegetable oils and polyethylene glycols. Additionally, lubricating agents such as magnesium stearate, sodium lauryl sulfate, sodium stearyl fumarate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives or buffers, which enhance the shelf life or effectiveness of crystalline anhydrous Compound 1, Form 1.

The compositions of this invention may be in a variety of forms. These include, for example, tablets, capsules, pills, liquid, semi-solid and solid dosage forms, such as liquid solutions (e.g., injectable and infusible solutions), dispersions or suspensions, powders, liposomes and suppositories. The form depends on the intended mode of administration and therapeutic application.

Oral administration of a solid dosage form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dosage form is sub-lingual, such as, for example, a lozenge. In such solid dosage forms, crystalline anhydrous Compound 1, Form 1 is ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the invention comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using suitable dispersing, wetting agents, and/or suspending agents.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, crystalline anhydrous Compound 1, Form 1 is administered by intravenous infusion or injection. In yet another embodiment, crystalline anhydrous Compound 1, Form 1 is administered by intramuscular or subcutaneous injection.

In another embodiment, the invention comprises a topical dosage form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When crystalline anhydrous Compound 1, Form 1 is administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein crystalline anhydrous Compound 1, Form 1 is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration or administration by inhalation, crystalline anhydrous Compound 1, Form 1 is conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone (e.g., crospovidone); amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250 or 500 milligrams of the active ingredient for the symptomatic adjustment of the dosage to the subject. A compositions typically contains from about 0.01 mg to about 500 mg of the active ingredient, or in another embodiment, from about 1 mg to about 100 mg of the active ingredient.

Intravenously, doses may range from about 0.01 to about 10 mg/kg/minute during a constant rate infusion.

Liposomes containing crystalline anhydrous Compound 1, Form 1 may be prepared by methods known in the art, such as described in U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Crystalline anhydrous Compound 1, Form 1 may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. A compound for intravenous administration is generally placed into a container having a sterile access port, for example, an intravenous solution bag or vial having a stopper pierceable by a hypodermic injection needle.

Suitable emulsions may be prepared using commercially available fat emulsions, such as a lipid emulsion comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

For example, the emulsion compositions can be those prepared by mixing a compound of the invention with a lipid emulsion comprising soybean oil or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

In one embodiment, provided herein is a pharmaceutical composition comprising crystalline anhydrous Compound 1, Form 1 which is formulated for oral administration. In one embodiment, the pharmaceutical composition formulated for oral administration is in the form of a tablet or capsule.

In one embodiment, the pharmaceutical composition formulated for oral administration is in the form of a tablet. In one embodiment, the tablet formulation comprises from about 1 to about 100 mg of crystalline anhydrous Compound 1, Form 1. In one embodiment, the tablet formulation comprises about 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg of crystalline anhydrous Compound 1, Form 1. In one embodiment, the tablet formulation comprises about 5 mg, 25 mg, 50 mg or 100 mg of crystalline anhydrous Compound 1, Form 1. In one embodiment, the tablet formulation comprises about 5 mg of crystalline anhydrous Compound 1, Form 1. In one embodiment, the tablet formulation comprises about 25 mg of crystalline anhydrous Compound 1, Form 1. In one embodiment, the tablet formulation comprises about 50 mg of crystalline anhydrous Compound 1, Form 1. In one embodiment, the tablet formulation comprises about 100 mg of crystalline anhydrous Compound 1, Form 1.

In one embodiment, provided herein is a pharmaceutical composition which is formulated for oral administration, comprising crystalline anhydrous Compound 1, Form 1, microcrystalline cellulose, dibasic calcium phosphate anhydrous, crospovidone, and sodium stearyl fumarate. In one embodiment, the pharmaceutical composition is formulated as a tablet.

In one embodiment, provided herein is a pharmaceutical composition comprising crystalline anhydrous Compound 1, Form 1 which is formulated for oral administration, wherein said pharmaceutical composition is selected from Formula 1 and Formula 2 shown below. Formulation 1 may be used to prepare a 5 mg tablet by compressing 100 mg of the Formulation 1 blend or a 25 mg tablet by compressing 500 mg of the Formulation 1 blend. Formulation 2 may be used to prepare a 100 mg tablet by compressing 800 mg of the Formulation 2 blend.

Formulation 1

| | Amount |
|---|---|
| Component | (w/w %) |
| Crystalline anhydrous Compound 1, Form 1 | 5.000 |
| Microcrystalline cellulose (Avicel PH102) | 60.000 |
| Dibasic calcium phosphate anhydrous (A-TAB) | 30.000 |
| Crospovidone Type B (Kollidon CL-SF) | 3.000 |
| Sodium stearyl fumarate (PRUV) | 2.000 |
| | TOTAL: 100.000 |

Formulation 2

| | Amount |
|---|---|
| Component | (w/w %) |
| Crystalline anhydrous Compound 1, Form 1 | 12.500 |
| Microcrystalline cellulose (Avicel PH102) | 55.000 |
| Dibasic calcium phosphate anhydrous (A-TAB) | 27.500 |
| Crospovidone Type B (Kollidon CL-SF) | 3.000 |
| Sodium stearyl fumarate (PRUV) | 2.000 |
| | TOTAL: 100.000 |

### Methods of Treatment

Crystalline anhydrous Compound 1, Form 1 may be useful for treating diseases and disorders which can be treated with a BRAF kinase inhibitor, such as BRAF-associated diseases and disorders, e.g., BRAF-associated tumors. The ability of Compound 1 to act as a BRAF inhibitor may be demonstrated by the enzyme assay described in Example A1, the cell assay described in Example A2, the cellular assay described in Example A3, and the proliferation assay described in Example A4.

Accordingly, in one embodiment, the invention further provides a method of treating a BRAF-associated tumor, in a subject in need thereof, comprising administering to the subject a therapeutically effective amount of crystalline anhydrous Compound 1, Form 1 or a pharmaceutical composition thereof.

The invention further provides crystalline anhydrous Compound 1, Form 1 for use in the treatment of a BRAF-associated disease or disorder.

The invention further provides use of crystalline anhydrous Compound 1, Form 1 in the manufacture of a medicament for the treatment of a BRAF-associated disease or disorder.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. However, “treat” or “treatment” can also include therapeutic measures (e.g., inhibition of BRAF kinase in a BRAF-associated tumor) that temporarily worsen the appearance and/or symptoms of the subject. As used herein, the terms “treating” and “treating” when referring, e.g., to the treatment of a cancer, are not intended to be absolute terms. For example, “treatment of a tumor” and “treating cancer”, as used in a clinical setting, is intended to include obtaining beneficial or desired clinical results and can include an improvement in the condition of a subject having cancer. Beneficial or desired clinical results include, but are not limited to, one or more of the following: reducing the proliferation of (or destroying) neoplastic or cancerous cells, inhibiting metastasis of neoplastic cells, a decrease in metastasis in a subject, shrinking or decreasing the size of a tumor, change in the growth rate of one or more tumor(s) in a subject, an increase in the period of remission for a subject (e.g., as compared to the one or more metric(s) in a subject having a similar cancer receiving no treatment or a different treatment, or as compared to the one or more metric(s) in the same subject prior to treatment), decreasing symptoms resulting from a disease, increasing the quality of life of those suffering from a disease (e.g., assessed using FACT-G or EORTC-QLQC30), decreasing the dose of other medications required to treat a disease, delaying the progression of a disease, and/or prolonging survival of subjects having a disease. “Treatment” can also mean prolonging survival as compared to expected survival if not receiving treatment, for example, an increase in overall survival (OS) compared to a subject not receiving treatment as described herein, and/or an increase in progression-free survival (PFS) compared to a subject not receiving treatment as described herein.

As used herein, the term “subject” refers to any animal, including mammals such as mice, rats, other rodents, rabbits, dogs, cats, swine, cattle, sheep, horses, primates, and humans. In some embodiments, the subject is a human. In some embodiments, the subject is suspected of having a BRAF-associated tumor. In some embodiments, the subject is a human. In some embodiments, the human subject is an adult subject. In some embodiments, the human subject is a pediatric subject.

“Ameliorating” or “amelioration” means a lessening or improvement of one or more symptoms as compared to not administering a treatment. “Ameliorating” also includes shortening or reduction in duration of a symptom.

The term “BRAF-associated” with respect to a disease or disorder as used herein refers to diseases or disorders associated with or having one or more BRAF mutation selected from Class I, Class II and Class III BRAF mutations. Non-limiting examples of a BRAF-associated disease or disorder include, for example, BRAF-associated tumors.

The phrase “BRAF mutation” refers to a genetic mutation (e.g., a chromosomal translocation that results in one or more mutations in a BRAF gene that results in the expression of a BRAF protein with one or more point mutations as compared to a wild type BRAF protein), or an alternative spliced version of a BRAF mRNA that results in a BRAF protein having a deletion of at least one amino acid in the BRAF protein as compared to the wild-type BRAF protein (i.e., a splice variant). Non-limiting examples of BRAF mutations include Class I BRAF mutations (e.g., BRAF V600 mutations, e.g., BRAF V600E and BRAF V600K), Class II BRAF mutations (e.g., BRAF non-V600 mutations and BRAF splice variants), and BRAF Class Ill mutations.

The term “Class I BRAF mutations” refers to BRAF V600 mutations which signal as Ras-independent active monomers. Examples include BRAF V600E and BRAF V600K mutations.

The term “Class II BRAF mutations” includes (i) BRAF non-V600 mutations which function as RAS-independent activated dimers of BRAF and (ii) BRAF splice variants which are dependent on dimerization for activity in a RAS-independent fashion.

Examples of BRAF non-V600 (Class II) mutations include G469A, G469R, G469V, K601E, K601N, K601T, L597Q and L597V. In one embodiment, the BRAF non-V600 mutation is G469A.

The term “BRAF splice variant” refers to aberrantly spliced BRAF V600E isoforms. BRAF splice variants are BRAF V600E resistance mutations that lack exons encoding part of the RAS-binding domain and exhibit enhanced dimerization in cells with low levels of RAS activation (Poulikakos et al., Nature, 480(7377):387-390. Examples of Class II BRAF V600E splice variants include those lacking exons 4-8 (also known as p61 BRAF(V600E)), exons 4-10, exons 2-8 or exons 2-10. In one embodiment, the resistance mutation is p61BRAF(V600E).

The term “resistance mutation” refers to a mutation in a BRAF V600E mutation that results after exposure of the BRAF V600E mutant to a BRAF inhibitor, either alone or in combination with another anticancer agent such as a MEK inhibitor. Tumors having resistance mutations become less sensitive to (e.g., resistant to treatment with) BRAF inhibitors. In one embodiment, the resistance mutation results after exposure to vemurafenib. In one embodiment, the resistance mutation results after exposure to encorafenib.

The term “Class III BRAF mutations” refers to BRAF non-V600 mutations which function as RAS-dependent activated dimers of BRAF and/or CRAF. Non-limiting examples of BRAF Class Ill mutations include G466A, G466E, G466R, G466V, D594A, D594E, D594G, D594H, G594N, D287H, V549L, S467A, S467E, S467L, G469E, N581S, N5811, F595L, G596A, G596C, G596D, G596R, and K483M.

The term “BRAF fusion” refers to a BRAF gene translocation that results in the expression of a fusion protein. In one embodiment, a BRAF-associated tumor or BRAF-associated cancer has one or more BRAF fusions that lead to constitutive kinase activation and transformation, including but not limited to KIAA11549-BRAF, MKRN1-BRAF, TRIM24-BRAF, AGAP3-BRAF, ZC3HAV1-BRAF, AKAP9-BRAF, CCDC6-BRAF, AGK-BRAF, EPS15-BRAF, NUP214-BRAF, ARMC10-BRAF, BTF3L4-BRAF, GHR-BRAF, ZC3HAV1-BRAF, ZNF767-BRAF, CCDC91-BRAF, DYNC112-BRAF, ZKSCAN1-BRAF, GTF21-BRAF, MZT1-BRAF, RAD18-BRAF, CUX1-BRAF, SLC12A7-BRAF, MYRIP-BRAF, SND1-BRAF, NUB1-BRAF, KLHL7-BRAF, TANK-BRAF, RBMS3-BRAF, STRN3-BRAF, STK35-BRAF, ETFA-BRAF, SVOPL-BRAF, JHDM1D-BRAF, or BCAP29-BRAF.

The term “BRAF-associated tumor” or “BRAF-associated cancer” as used herein to tumors or cancers associated with or having a BRAF mutation and includes tumors having one or more BRAF mutations selected from Class I BRAF, Class II BRAF mutations and Class Ill BRAF mutations. BRAF-associated tumors include both benign BRAF-associated tumors and malignant BRAF-associated tumors (i.e., BRAF-associated cancers).

The term “tumor” as used herein refers to an abnormal growth of tissue that arises from uncontrolled usually rapid cellular proliferation. The tumor may be a benign tumor (non-cancerous) or a malignant tumor (i.e., cancer). The tumor may be a solid tumor or a liquid tumor (i.e., a hematologic tumor, also known as blood cancer).

The term “wild type” describes a nucleic acid (e.g., a BRAF gene or a BRAF mRNA) that is typically found in a subject that does not have a disease or disorder related to the reference nucleic acid or protein.

The term “wild type BRAF” describes a BRAF nucleic acid (e.g., a BRAF gene or a BRAF mRNA) or a BRAF protein that is found in a subject that does not have a BRAF-associated disease, e.g., a BRAF-associated cancer (and optionally also does not have an increased risk of developing a BRAF-associated disease and/or is not suspected of having a BRAF-associated disease), or is found in a cell or tissue from a subject that does not have a BRAF-associated disease, e.g., a BRAF-associated cancer (and optionally also does not have an increased risk of developing a BRAF-associated disease and/or is not suspected of having a BRAF-associated disease).

In one embodiment, provided herein is a method of treating a BRAF-associated tumor in a subject in need of such treatment, the method comprising administering to the subject a therapeutically effective amount of crystalline anhydrous Compound 1, Form 1.

As used herein, a “therapeutically effective amount” of a compound is an amount sufficient to achieve any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include providing a therapeutic effect can include reducing the size of a tumor, inhibiting (e.g., slowing, to some extent, preferably stopping) tumor progression, inhibiting (e.g., slowing, to some extent, preferably stopping) tumor growth, inhibiting (e.g., slowing, to some extent, preferably stopping) tumor invasiveness, and/or inhibiting (e.g., slowing, to some extent, preferably stopping) tumor metastasis. The skilled person understands that tumor progression in human subjects can be determined by a variety of methods. For example, the size of a tumor close to the skin can be measured by establishing the width and depth of the tumor with calipers, and then calculating the tumor volume. Less accessible tumors, such as lung and CNS cancers can be measured by observation of the images obtained from Magnetic Resonance Imaging (MRI) scanning. CNS tumors, such as brain tumors, can be measured by a combination of MRI scanning and by monitoring neurological performance. Growth of a brain tumor is typically associated with decreasing neurological performance. Providing a therapeutic effect also includes prolonging survival of a subject or subject beyond that expected in the absence of treatment and/or relieving to some extent (or preferably eliminating) one or more signs or symptoms associated with cancer. In one embodiment, treatment of a subject or subject with a compound or combination according to an invention prolongs survival beyond that expected in the absence of treatment by 1 or months, e.g., by 3 or more months, e.g., by 6 or more months, e.g., by 1 or more years, e.g., by 2 or more years, e.g., by 3 or more years, e.g., by 5 or more years, e.g., by 10 or more years. Providing a therapeutic effect also includes reducing the number of cancer cells. Providing a therapeutic effect also includes eliminating cancer cells. Providing a therapeutic effect also includes tumor mass reduction. Providing a therapeutic effect also includes causing a cancer to go into remission. A therapeutically effective amount can be administered in one or more administrations. For purposes of this invention, dosage therapeutically effective amount of a compound, or pharmaceutical composition thereof is an amount sufficient to accomplish prophylactic or therapeutic treatment either directly or indirectly. As is understood in the clinical context, dosage therapeutically effective amount of a compound or pharmaceutical composition thereof may be achieved in conjunction with another therapy.

A “therapeutically effective amount” may be considered in the context of administering one or more therapies (e.g., one or more anticancer agents), and a single agent may be considered to be given in a therapeutically effective amount if, in conjunction with one or more other agents, a desirable result may be or is achieved. In reference to the treatment of a tumor, a therapeutically effective amount may also refer to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in subjects with these specific tumors, which may be measured as prolongation of the time before disease progression.

In one embodiment, a subject treated according to any of the methods disclosed herein may be assessed according to one or more standard response assessment criteria known in the art, including RECIST (Response Evaluation Criteria in Solid Tumors, e.g., RECIST version 1.0, RECIST version 1.1, and modified RECIST 1.1 (mRECIST 1.1)), RANO-BM (Response Assessment in Neuro-Oncology Brain Metastases), Macdonald, RANO-LMD, and NANO (Neurologic Assessment in Neuro-Oncology). In one embodiment of any of said criteria, the tumor is assessed by an imaging study (e.g., MRI, CT, MDCT or PET). In one embodiment the treatment response is assessed in accordance with RECIST version 1.1, wherein: complete response (CR) is defined as the complete disappearance of all tumor lesions; partial response (PR) is defined as a reduction in the sum of tumor measurements by at least 30%; progressive disease (PD) is defined as at least 20% increase in the sum of tumor measurements (wherein the development of new lesions or substantial progression of non-target lesions is also was defined as PD) wherein an increase of at least 5 mm from baseline is evaluated as PD; and stable disease (SD) is defined as neither sufficient shrinkage to qualify for PR nor sufficient increase to qualify for PD, taking as reference the smallest sum diameters while on treatment. In one embodiment, assessments include intracranial response (assessed as per modified RECIST using gadolinium enhanced MRI), extracranial response, global response rate, disease control rate (DCR), duration of response (DOR), progression free survival (PFS), and overall survival (OS).

In one embodiment of any of the methods of use described herein, the BRAF-associated tumor is a solid tumor. In some embodiments, the tumor is intracranial. In some embodiments, the tumor is extracranial. In some embodiments of any of the methods of uses described herein, the BRAF-associated tumor is a malignant BRAF-associated tumor (i.e., a BRAF-associated cancer). In some embodiments of any of the methods of use described herein, the cancer is melanoma, colon cancer, colorectal cancer, lung cancer (e.g., small cell lung cancer or non-small cell lung cancer), thyroid cancer (e.g., papillary thyroid cancer, medullary thyroid cancer, differentiated thyroid cancer, recurrent thyroid cancer, or refractory differentiated thyroid cancer), breast cancer, bladder cancer, ovarian cancer (ovary carcinoma), cancer of the CNS (including gliomas and LMDs), bone cancer, cancer of the anus, anal canal, or anorectum, angiosarcoma, adenoid cystic carcinoma, appendiceal cancer, cancer of the eye, bile duct cancer (cholangiocarcinoma), cervical cancer, ductal carcinoma in situ, endometrial cancer, gallbladder, hepatobiliary cancer, hepato-pancreato-biliary carcinoma, head and neck squamous cell carcinoma, oral cancer, oral cavity cancer, leukemia, lip cancer, oropharyngeal cancer, cancer of the nose, nasal cavity or middle ear, cancer of the vulva, esophageal cancer, esophagogastric cancer, cervical cancer, gastrointestinal carcinoid tumor, gastrointestinal neuroendocrine cancer, hypopharynx cancer, kidney cancer, larynx cancer, liver cancer, nasopharynx cancer, non-Hodgkin's lymphoma, peripheral nervous system cancers (e.g., neuroblastoma), neuroendocrine cancer, pancreatic cancer, peritoneum, plasma cell neoplasm, omentum, and mesentery cancer, pharynx cancer, prostate cancer, renal cancer (e.g., renal cell carcinoma (RCC)), small bowel cancer, small intestine cancer, soft tissue sarcoma, stomach cancer, testicular cancer, uterine cancer, ureter cancer, or urinary bladder cancer, and metastatic cancers thereof.

In one embodiment, the BRAF-associated tumor is a BRAF-associated cancer selected from CNS cancer (i.e., a metastatic brain cancer or a primary brain tumor), melanoma, colorectal cancer, thyroid cancer, non-small cell lung cancer, ovarian cancer, and renal cell carcinoma.

In one embodiment, the BRAF-associated tumor is an extracranial BRAF-associated cancer selected from melanoma, colorectal cancer, thyroid cancer, non-small cell lung cancer, ovarian cancer, and neuroblastoma. In some embodiments, the BRAF-associated cancer is melanoma. In some embodiments, the BRAF-associated cancer is colorectal cancer. In some embodiments, the BRAF-associated cancer is thyroid cancer. In some embodiments, the BRAF-associated cancer is non-small cell lung cancer. In some embodiments, the BRAF-associated cancer is ovarian cancer. In some embodiments, the BRAF-associated cancer is neuroblastoma.

In one embodiment, the BRAF-associated tumor is a cancer having a BRAF Class I mutation. In some embodiments, the BRAF-associated cancer is a cancer having a BRAF V600E or BRAF V600K mutation. In some embodiments, the BRAF-associated cancer having a BRAF V600E or BRAF V600K mutation is selected from melanoma, colorectal cancer, thyroid cancer, non-small cell lung cancer, ovarian cancer, renal cell carcinoma, and metastatic cancers thereof, and primary brain tumors. In some embodiments, the BRAF-associated cancer having a BRAF V600E or BRAF V600K mutation is a CNS tumor. In some embodiments, the CNS tumor is a malignant tumor (a CNS cancer). In some embodiments, the malignant tumor is a metastatic CNS cancer. In some embodiments, the metastatic CNS cancer is selected from metastatic melanoma, metastatic colorectal cancer, metastatic non-small cell lung cancer, metastatic thyroid cancer, and metastatic ovarian cancer. In some embodiments, the CNS tumor is a primary brain tumor. In some embodiments, the CNS tumor is intracranial LMD or extracranial LMD.

In one embodiment, the BRAF-associated tumor is a cancer having a BRAF Class II mutation. In one embodiment, the cancer having a BRAF Class II mutation is selected from lung cancer (e.g., non-small cell lung cancer), melanoma, colorectal cancer, breast cancer, pancreatic cancer, thyroid cancer, prostate cancer, adenoid cystic carcinoma, appendiceal cancer, small intestine cancer, gastrointestinal neuroendocrine cancer, head and neck squamous cell carcinoma, angiosarcoma, bladder cancer, plasma cell neoplasm, hepatobiliary cancer, hepato-pancreato-biliary carcinoma, ovarian cancer, endometrial cancer, neuroendocrine cancer, cholangiocarcinoma, esophagogastric cancer, soft tissue sarcoma, leukemia, non-Hodgkin's lymphoma, and CNS cancers (e.g., gliomas). In one embodiment, the cancer has a BRAF G469A mutation.

In one embodiment, the BRAF-associated tumor is a cancer having a BRAF Class III mutation. In one embodiment, the cancer having a BRAF class III mutation is selected from melanoma, small bowel cancer, colorectal cancer, non-small cell lung cancer, endometrial cancer, cervical cancer, leukemia, bladder cancer, non-Hodgkin's lymphoma, glioma, ovarian cancer, prostate cancer, hepatobiliary cancer, esophagogastric cancer, soft tissue sarcoma, and breast cancer. In one embodiment, the cancer has a BRAF G466V or BRAF D594G mutation. In one embodiment, the cancer has a BRAF G466V mutation. In one embodiment, the cancer has a BRAF D594G mutation.

In one embodiment, the BRAF-associated tumor has a BRAF-fusion protein, wherein the tumor is breast carcinoma (e.g., breast invasive ductal carcinoma) colorectal carcinoma (e.g., colon adenocarcinoma), esophageal carcinoma (e.g., esophagus adenocarcinoma), glioma (e.g., brain desmoplastic infantile ganglioglioma, brain pilocytic astrocytoma, brain pleomorphic xanthoastrocytoma, spinal cord low-grade glioma (NOS), anaplastic oligodendroglioma, anaplastic ganglioglioma), head & neck carcinoma (e.g., head and neck neuroendocrine carcinoma), lung carcinoma (e.g., lung adenocarcinoma, lung non-small-cell lung cancer (NOS)), melanoma (e.g., cutaneous melanoma Spitzoid, mucosal melanoma non-Spitzoid, cutaneous melanoma Spitzoid, unknown primary melanoma, cutaneous melanoma non-Spitzoid), pancreatic carcinoma (e.g., adenocarcinoma, pancreas acinar cell carcinoma), prostatic carcinoma (e.g., prostate acinar adenocarcinoma), sarcoma (malignant solid fibrous tumor), thyroid carcinoma (thyroid papillary carcinoma), unknown primary carcinoma (e.g., unknown primary, adenocarcinoma), pleura mesothelioma, rectum adenocarcinoma, uterus endometrial carcinoma (e.g., uterus endometrial adenocarcinoma (NOS)) or ovary serous carcinoma.

The terms “metastasis” and “metastatic” are art known terms that refer to the spread of cancer cells from the place where they first formed (the primary site) to one or more other sites in a subject (one or more secondary sites). In metastasis, cancer cells break away from the original (primary) tumor, travel through the blood or lymph system, and form a new tumor (a metastatic tumor) in other organs or tissues of the body. The new, metastatic tumor includes the same or similar cancer cells as the primary tumor. At the secondary site, the tumor cell may proliferate and begin the growth or colonization of a secondary tumor at this distant site.

The term “metastatic cancer” (also known as “secondary cancer”) as used herein refers to a type of cancer that originates in one tissue type, but then spreads to one or more tissues outside of the (primary) cancer's origin. Metastatic brain cancer refers to cancer in the brain, i.e., cancer which originated in a tissue other than the brain and has metastasized to the brain.

In one embodiment, the BRAF-associated tumor is a CNS tumor. In one embodiment, the BRAF-associated CNS tumor is a malignant BRAF-associated CNS tumor (i.e., “a BRAF-associated CNS cancer”). The term “CNS cancer” or “cancer of the CNS” or as used interchangeably herein refers to a cancer (i.e., a malignant tumor) of the CNS, including cancers of the brain (also known as intracranial tumors), cancers of the spinal cord, and cancers of the meninges surrounding the brain and spinal cord. The term “BRAF-associated CNS cancer” refers to CNS cancer associated with or having a BRAF mutation. Cancers of the CNS include metastatic brain cancers and primary brain tumors.

Leptomeningeal metastases (leptomeningeal disease (LMD)) represent a subset of CNS metastases that grow in the lining of the brain or spine and/or in the cerebrospinal fluid (CSF), or leptomeningeal carcinomatosis. In mammals, the meninges are the dura mater, the arachnoid mater, and the pia mater. CSF is located in the subarachnoid space between the arachnoid mater and the pia mater. The arachnoid and pia mater together are sometimes called the leptomeninges. When LMD occurs in the leptomeninges and/or CSF surrounding the spinal cord, it may be referred to as “extracranial LMD”. When LMD occurs in the leptomeninges and/or CSF of the brain, it may be referred to as “intracranial LMD”. Since LMD cancer cells can be suspended in the CSF, they can quickly spread throughout the CNS. As a result, LMD has a poor prognosis, with survival typically measured in months. In one embodiment, the metastatic cancer is BRAF-associated LMD. In one embodiment, the metastatic cancer is intracranial BRAF-associated LMD. In one embodiment, the metastatic cancer is extracranial BRAF-associated LMD. BRAF-associated cancers with the highest incidences of leptomeningeal metastases are lung cancer and melanoma. In one embodiment the BRAF-associated LMD is LMD derived from melanoma metastases (i.e., the LMD is metastatic melanoma). In one embodiment the BRAF-associated LMD is LMD derived from colorectal cancer metastases (i.e., the LMD is metastatic colorectal cancer). In one embodiment the BRAF-associated LMD is LMD derived from non-small cell lung cancer metastases (i.e., the LMD is metastatic non-small cell lung cancer).

In one embodiment, the BRAF-associated CNS tumor is a BRAF-associated primary brain tumor. In one embodiment, the primary brain tumor is a malignant primary brain tumor. In one embodiment, the primary brain tumor is a benign primary brain tumor. In one embodiment, the primary brain tumor has Class I mutation. In one embodiment the primary brain tumor has a BRAF V600 mutation. In one embodiment the primary brain tumor has a BRAF V600E or BRAF V600K mutation. In one embodiment, the primary brain tumor has a Class II mutation. In one embodiment, the primary brain tumor has a Class II mutation selected from G469A, G469R, G469V, K601E, K601N, K601T, L597Q and L597V. In one embodiment, the primary brain tumor has a G469A mutation.

“Primary brain tumors” are tumors that start in the brain or spine and are known collectively as gliomas. The term “glioma” is used to describe tumors that originate in glial cells present in the CNS. According to the WHO classification of brain tumors, gliomas are graded by the cell activity and aggressiveness on a scale including Grade I (benign CNS tumors) and Grades II to IV (malignant CNS tumors):

Grade I glioma (Pilocytic astrocytoma): typically occurs in children in the cerebellum or brainstem, and occasionally in the cerebral hemispheres, and are slow growing. Grade I can occur in adults. Although they are benign (WHO grade I), the difficulty in curing this disease makes their growth malignant in behavior with high morbidity rates (Rostami, Acta Neurochir (Wien). 2017; 159(11): 2217-2221).

Grade II glioma (Low-grade gliomas): includes astrocytoma, oligodendroglioma, and mixed oligoastrocytma. Grade II gliomas typically occur in young adults (20s-50s) and are most often found in the cerebral hemispheres. Due to the infiltrative nature of these tumors, recurrences may occur. Some grade II gliomas recur and evolve into more aggressive tumors (grade III or IV).

Grade III glioma (Malignant glioma): includes anaplastic astrocytoma, anaplastic oligodendroglioma, and anaplastic mixed oligoastrocytoma. Grade III tumors are aggressive, high-grade cancers and invade nearby brain tissue with tentacle-like projections, making complete surgical removal more difficult.

Grade IV gliomas: includes Glioblastoma multiforme (GBM) and gliosarcoma; (GBM) is a malignant glioma. GBM is the most aggressive and most common primary brain tumor. Glioblastoma multiforme usually spreads quickly and invades other parts of the brain, with tentacle-like projections, making complete surgical removal more difficult. Gliosarcoma is a malignant cancer and is defined as a glioblastoma consisting of gliomatous and sarcomatous components.

In one embodiment, the BRAF-associated primary brain tumor is a glioma. In some embodiments, the BRAF-associated primary brain tumor is a glioma having a Class I mutation. In some embodiments, the BRAF-associated primary brain tumor is a glioma having a Class II mutation.

Benign primary brain tumors can cause severe pain, permanent brain damage and death, and in some cases, become malignant. Non-limiting examples of benign primary brain tumors include Grade I gliomas, papillary craniopharyngiomas, meningioma (including rhabdoid meningioma), atypical teratoid/rhabdoid tumors, and dysembryoplastic neuroepithelial tumor (DNT), pilocytic astrocytoma, oligodendroglioma, mixed oligoastrocytoma, anaplastic astrocytoma, anaplastic oligodendroglioma, anaplastic mixed oligoastrocytoma, diffuse astrocytoma, ependymoma, a pleomorphic xanthoastrocytoma (PXA), a ganglioglioma, a gliosarcoma, or an anaplastic ganglioglioma. In one embodiment, the BRAF-associated tumor is a benign primary brain tumor.

In one embodiment, the BRAF-associate cancer is a peripheral nervous system cancer. In one embodiment, the peripheral nervous system cancer is neuroblastoma.

The ability to determine whether Compound 1 may be suitable for treating a CNS cancer may be determined, for example, by identifying if Compound 1 is a substrate of an efflux transporter and/or measuring the cell permeability and/or measuring the free brain-to-free plasma ratio, as described hereinbelow in Examples B and C.

In one embodiment, crystalline anhydrous Compound 1, Form 1 is administered combination with one or more different forms of treatment to treat a subject with a BRAF-associated tumor. For example, crystalline anhydrous Compound 1, Form 1 may be used in combination with one or more additional anticancer therapies independently selected from surgery, radiotherapy, and one or more anticancer agents. In one embodiment, treatment of a subject having a BRAF-associated tumor with crystalline anhydrous Compound 1, Form 1 in combination with one or more additional therapies, e.g., surgery, radiotherapy, and/or an anticancer agent, can have increased therapeutic efficacy as compared to treatment of the same subject or a similar subject with crystalline anhydrous Compound 1, Form 1 as a monotherapy.

Accordingly, in one embodiment, provided herein are methods of treating a subject having a BRAF-associated tumor (e.g., any of the BRAF-associated tumors described herein) that include: administering to the subject (i) a therapeutically effective amount of a compound of crystalline anhydrous Compound 1, Form 1 as a monotherapy, or (ii) a therapeutically effective amount of crystalline anhydrous Compound 1, Form 1 in combination with one or more additional anticancer therapies.

Also provided herein is crystalline anhydrous Compound 1, Form 1 for use in combination with one or more additional anticancer therapies.

Examples of additional anticancer therapies that can be used in combination with crystalline anhydrous Compound 1, Form 1, according to any of the above-described methods include but are not limited to, MEK inhibitors, EGFR inhibitors, inhibitors of HER2 and/or HER3, Axl inhibitors, PI3K inhibitors, and SOS1 inhibitors), signal transduction pathway inhibitors, checkpoint inhibitors, modulators of the apoptosis pathway, cytotoxic chemotherapeutics, angiogenesis-targeted therapies, and immune-targeted agents including immunotherapy.

In one embodiment, crystalline anhydrous Compound 1, Form 1 is administered in combination with an additional anticancer agent which is a MEK inhibitor. In one embodiment, the MEK inhibitor is selected from binimetinib, trametinib, cobimetinib, selumetinib, pimasertib, refametinib, mirdametinib, 2-(2-chloro-4-iodophenylamino)-N-(cyclopropylmethoxy)-3,4-difluorobenzamide (CI-1040), 3-[2(R),3-dihydroxypropyl]-6-fluoro-5-(2-fluoro-4-iodophenylamino)-8-methylpyrido[2,3-d]pyrimidine-4,7(3H,8H)-dione (TAK-733), and pharmaceutically acceptable salts thereof. In one embodiment the MEK inhibitor is binimetinib or a pharmaceutically acceptable salt thereof.

In one embodiment, crystalline anhydrous Compound 1, Form 1 is administered in combination with an additional anticancer agent which is an EGFR inhibitor. Non-limiting examples of EGFR inhibitors include cetuximab (Erbitux®), panitumumab (Vectibix®), osimertinib (merelectinib, Tagrisso®), erlotinib (Tarceva®), gefitinib (Iressa®), necitumumab (Portrazza™) neratinib (Nerlynx®), lapatinib (Tykerb®), vandetanib (Caprelsa®), brigatinib (Alunbrig®). In one embodiment, the EGFR inhibitor is cetuximab.

In one embodiment, crystalline anhydrous Compound 1, Form 1 is administered in combination with an additional anticancer agent selected from a MEK inhibitor (e.g., any of the MEK inhibitors disclosed herein) and an EGFR inhibitor (e.g., any of the EGFR inhibitors disclosed herein).

In one embodiment, crystalline anhydrous Compound 1, Form 1 is administered in combination with a MEK inhibitor which is binimetinib or a pharmaceutically acceptable salt thereof, and an EGFR inhibitor which is cetuximab.

In one embodiment, crystalline anhydrous Compound 1, Form 1 is administered in combination with an additional anticancer agent which is a HER2 and/or HER3 inhibitor. Non-limiting examples of HER2 and/or HER3 inhibitors include lapatinib, canertinib, (E)-2-methoxy-N-(3-(4-(3-methyl-4-(6-methylpyridin-3-yloxy)phenylamino)quinazolin-6-yl)allyl)acetamide (CP-724714), sapitinib, 7-[[4-[(3-ethynylphenyl)amino]-7-methoxy-6-quinazolinyl]oxy]-N-hydroxy-heptanamide (CUDC-101), mubritinib, 6-[4-[(4-ethylpiperazin-1-yl)methyl]phenyl]-N-[(1R)-1-phenylethyl]-7H-pyrrolo[2,3-d]pyrimidin-4-amine (AEE788), irbinitinib (tucatinib), poziotinib, N-[4-[1-[4-(4-acetyl-1-piperazinyl)cyclohexyl]-4-amino-3-pyrazolo[3,4-d]pyrimidinyl]-2-methoxyphenyl]-1-methyl-2-indolecarboxamide (KIN001-111), 7-cyclopentyl-5-(4-phenoxyphenyl)-7H-pyrrolo[2,3-d]pyrimidin-4-ylamine (KIN001-051), 6,7-dimethoxy-N-(4-phenoxyphenyl)quinazolin-4-amine (KIN001-30), dasatinib, and bosutinib, and pharmaceutically acceptable salts thereof.

In one embodiment, crystalline anhydrous Compound 1, Form 1 is administered in combination with an additional anticancer agent which is an Axl inhibitor. Non-limiting examples of Axl inhibitors include bemcentinib, 2-(5-chloro-2-(4-((4-methylpiperazin-1-yl)methyl)phenylamino)pyrimidin-4-ylamino)-N,N-dimethylbenzenesulfonamide (TP-0903), 3-[2-[[3-fluoro-4-(4-methyl-1-piperazinyl)phenyl]amino]-5-methyl-7Hpyrrolo[2,3-d]pyrimidin-4-yl]-benzeneacetonitrile (SGI-7079), gilteritinib, bosutinib, cabozantinib, sunitinib, foretinib, amuvatinib, glesatinib, N-(4-((2-amino-3-chloropyridin-4-yl)oxy)-3-fluorophenyl)-4-ethoxy-1-(4-fluorophenyl)-2-oxo-1,2-dihydropyridine-3-carboxamide (BMS777607), merestinib, (Z)-3-((3-((4-(morpholinomethyl)-1H-pyrrol-2-yl)methylene)-2-oxoindolin-5-yl)methyl)thiazolidine-2,4-dione (S49076), and (R)—N-(3-fluoro-4-((3-((1-hydroxypropan-2-yl)amino)-1H-pyrazolo[3,4-b]pyridin-4-yl)oxy)phenyl)-3-(4-fluorophenyl)-1-isopropyl-2,4-dioxo-1,2,3,4-tetrahydropyrimidine-5-carboxamide, and pharmaceutically acceptable salts thereof.

In one embodiment, crystalline anhydrous Compound 1, Form 1 is administered in combination with an additional anticancer agent which is a SOS1 inhibitor. Non-limiting examples of SOS1 inhibitors include those disclosed in PCT Publication No. WO 2018/115380, which is incorporated herein by reference in its entirety.

In one embodiment, crystalline anhydrous Compound 1, Form 1 is administered in combination with an additional anticancer agent which is a PI3K inhibitor. Non-limiting examples include buparlisib (BKM120), alpelisib (BYL719), samotolisib (LY3023414), 8-[(1R)-1-[(3,5-difluorophenyl)amino]ethyl]-N,N-dimethyl-2-(morpholin-4-yl)-4-oxo-4H-chromene-6-carboxamide (AZD8186), tenalisib (RP6530), voxtalisib hydrochloride (SAR-245409), gedatolisib (PF-05212384), panulisib (P-7170), taselisib (GDC-0032), trans-2-amino-8-[4-(2-hydroxyethoxy)cyclohexyl]-6-(6-methoxypyridin-3-yl)-4-methylpyrido[2,3-d]pyrimidin-7(8H)-one (PF-04691502), duvelisib (ABBV-954), N2-[4-oxo-4-[4-(4-oxo-8-phenyl-4H-1-benzopyran-2-yl)morpholin-4-ium-4-ylmethoxy]butyryl]-L-arginyl-glycyl-L-aspartyl-L-serine acetate (SF-1126), pictilisib (GDC-0941), 2-methyl-1-[2-methyl-3-(trifluoromethyl)benzyl]-6-(morpholin-4-yl)-1H-benzimidazole-4-carboxylic acid (GSK2636771), idelalisib (GS-1101), umbralisib tosylate (TGR-1202), pictilisib (GDC-0941), copanlisib hydrochloride (BAY 84-1236), dactolisib (BEZ-235), 1-(4-[5-[5-amino-6-(5-tert-butyl-1,3,4-oxadiazol-2-yl)pyrazin-2-yl]-1-ethyl-1H-1,2,4-triazol-3-yl]piperidin-1-yl)-3-hydroxypropan-1-one (AZD-8835), 5-[6,6-dimethyl-4-(morpholin-4-yl)-8,9-dihydro-6H-[1,4]oxazino[4,3-e]purin-2-yl]pyrimidin-2-amine (GDC-0084) everolimus, rapamycin, perifosine, sirolimus, and temsirolimus, and pharmaceutically acceptable salts thereof.

In one embodiment, crystalline anhydrous Compound 1, Form 1 is administered in combination with an additional anticancer agent which is an immunotherapy.

In one embodiment, the immunotherapy is an antibody therapy (e.g., a monoclonal antibody, a conjugated antibody). In some embodiments, the antibody therapy is bevacizumab (Mvasti™, Avastin®), trastuzumab (Herceptin®), rituximab (MabThera™, Rituxan®), edrecolomab (Panorex), daratumuab (Darzalex®), olaratumab (Lartruvo™), ofatumumab (Arzerra®), alemtuzumab (Campath®), oregovomab, pembrolizumab (Keytruda®), dinutiximab (Unituxin®), obinutuzumab (Gazyva®), tremelimumab (CP-675,206), ramucirumab (Cyramza®), ublituximab (TG-1101), panitumumab (Vectibix®), elotuzumab (Empliciti™), necitumumab (Portrazza™), cirmtuzumab (UC-961), ibritumomab (Zevalin®), isatuximab (SAR650984), nimotuzumab, fresolimumab (GC1008), lirilumab (INN), mogamulizumab (Poteligeo®), ficlatuzumab (AV-299), denosumab (Xgeva®), ganitumab, urelumab, pidilizumab, amatuximab, blinatumomab (AMG103; Blincyto®) or midostaurin (Rydapt).

In one embodiment, the immunotherapy is an immune checkpoint inhibitor. In some embodiments, the immunotherapy includes one or more immune checkpoint inhibitors. In some embodiments, the immune checkpoint inhibitor is a CTLA-4 inhibitor, a PD-1 inhibitor or a PD-L1 inhibitor. In some embodiments, the CTLA-4 inhibitor is ipilimumab (Yervoy®) ortremelimumab (CP-675,206). In some embodiments, the PD-1 inhibitor is pembrolizumab (Keytruda®) or nivolumab (Opdivo®). In some embodiments, the PD-L1 inhibitor is atezolizumab (Tecentriq®), avelumab (Bavencio®) or durvalumab (Imfinzi™). In one embodiment, the PD-1 inhibitor is RN888 (sasanlimab).

In any of the above methods wherein crystalline anhydrous Compound 1, Form 1 is administered in combination with one or more anticancer agents, crystalline anhydrous Compound 1, Form 1 and the additional anticancer agent(s) are formulated as separate compositions or dosages such that they may be administered to a subject in need thereof separately, either concurrently or sequentially with variable intervening time limits, wherein such administration provides effective levels of the two or more compounds in the body of the subject.

### Embodiments (EB)

EB1. A crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 having a ¹⁹F solid state NMR spectrum comprising resonance values of −188.1 and −115.8 ppm±0.2 ppm.

EB2. The crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to embodiment EB1, having a ¹³C solid state NMR spectrum comprising resonance (ppm) values at 35.8, 57.5, 130.6 and 148.1 ppm±0.2 ppm.

EB3. The crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to embodiment EB1 or EB2, having a powder X-ray diffraction pattern measured using copper wavelength radiation comprising peaks, in terms of 2-theta, at 8.3, 11.5 and 16.1 degrees 2-theta±0.2 degrees 2-theta.

EB4. The crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to any one of embodiments EB1 to EB3, having a powder X-ray diffraction pattern measured using copper wavelength radiation comprising peaks, in terms of 2-theta, at 8.3, 11.5, 16.1, 22.9, and 23.6 degrees 2-theta (±0.2 degrees 2-theta)

EB5. The crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to any one of embodiments EB1 to EB4, having a Raman spectrum comprising one or more wavenumber (cm⁻¹) values at 1308, 1433, 1447, 1548 and 1608 cm⁻¹±2 cm⁻¹.

EB6. A pharmaceutical composition comprising the crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to any one of embodiments EB1 to EB5, and one or more pharmaceutically acceptable excipients.

EB7. The pharmaceutical composition according to embodiment EB6, further comprises microcrystalline cellulose, dibasic calcium phosphate anhydrous, crospovidone Type B and sodium stearyl fumarate.

EB8. A method of treating a BRAF-associated tumor in a subject in need thereof, comprising administering to the subject the crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to any one of embodiments EB1 to EB5.

EB9. The method according to embodiment EB8, wherein said BRAF-associated tumor has a BRAF Class II mutation.

EB10. The method according to embodiment EB8 or EB9, wherein said BRAF-associated tumor is a cancer selected from lung cancer, melanoma, colorectal cancer, breast cancer, pancreatic cancer, thyroid cancer, prostate cancer, adenoid cystic carcinoma, appendiceal cancer, small intestine cancer, head and neck squamous cell carcinoma, angiosarcoma, bladder carcinoma, plasma cell neoplasm, hepato-pancreato-biliary carcinoma, ovary carcinoma, neuroendocrine cancer, cholangiocarcinoma and CNS cancers.

EB11. The method according to embodiment EB8, wherein said BRAF-associated tumor has a BRAF Class I mutation.

EB12. The method according to embodiment EB11, wherein said BRAF-associated tumor is selected from melanoma, colorectal cancer, thyroid cancer, non-small cell lung cancer, ovarian cancer, renal cell carcinoma, and metastatic cancers thereof, and primary brain tumors.

EB13. The method according to any one of embodiments EB8 to EB12, wherein the method further comprises administering one or more additional anticancer agents.

EB14. The method according to embodiment EB13, wherein the additional anticancer agent is a MEK inhibitor.

EB15. The method of embodiment EB14, wherein the MEK inhibitor is binimetinib or a pharmaceutically acceptable salt thereof.

EB16. The method of embodiment EB13, wherein the additional anticancer agent is an EGFR inhibitor, wherein the EGFR inhibitor is cetuximab.

EB17. The method of embodiment EB13, wherein the additional anticancer agents are a MEK inhibitor and an EGFR inhibitor, wherein the MEK inhibitor is binimetinib or a pharmaceutically acceptable salt thereof, and the EGFR inhibitor is cetuximab.

EB18. The crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to any one of embodiments EB1 to EB5 for use as a medicament.

EB19. The crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to any one of embodiments EB1 to EB5 for use in the treatment of a BRAF-associated tumor.

EB20. Use of the crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to any one of embodiments EB1 to EB5 for the manufacture of a medicament for the treatment of a BRAF-associated tumor.

### EXAMPLES

The examples and preparations provided below further illustrate and exemplify particular embodiments of the invention. It is to be understood that the scope of the present invention is not limited by the scope of the following examples.

### General Method 1

Powder X-ray Diffraction (PXRD) method for characteristic peak determination Powder X-ray diffraction analysis was conducted using a Bruker AXS D8 Endeavor diffractometer equipped with a Copper (Cu) radiation source. The divergence slit was set at 15 mm continuous illumination. Diffracted radiation was detected by a PSD-Lynx Eye detector, with the detector PSD opening set at 2.99 degrees. The X-ray tube voltage and amperage were set to 40 kV and 40 mA respectively. Data was collected in the Theta-Theta goniometer at the Cu wavelength (CuKα̅=1.5418λ) from 3.0 to 40.0 degrees 2-Theta using a step size of 0.01 degrees and a step time of 1.0 second. The antiscatter screen was set to a fixed distance of 3.0 mm. Samples were rotated at 15/min during collection. Samples were prepared by placing them in a silicon low background sample holder and rotated during collection. Data were collected using Bruker DIFFRAC Plus software and analysis was performed by EVA diffract plus software. The PXRD data file was not processed prior to peak searching. Using the peak search algorithm in the EVA software, peaks selected with a threshold value of 1 were used to make preliminary peak assignments. To ensure validity, adjustments were manually made; the output of automated assignments was visually checked, and peak positions were adjusted to the peak maximum. Peaks with relative intensity of >3% were generally chosen. Typically, the peaks which were not resolved or were consistent with noise were not selected. A typical error associated with the peak position from PXRD stated in USP up to +/−0.2° 2-Theta (USP-941).

### General Method 2

### Solid State NMR (ssNMR) Spectroscopy

Solid-state NMR (ssNMR) analysis was conducted on a CPMAS probe positioned into a Bruker-BioSpin Avance III 500 MHz (¹H frequency) NMR spectrometer. Material was packed into a 4 mm ZrO₂ rotor. A magic angle spinning rate of 15 kHz was used. Spectra were collected at ambient temperature (temperature uncontrolled).

¹³C ssNMR spectra were collected using a proton decoupled cross-polarization magic angle spinning (CPMAS) experiment. A phase modulated proton decoupling field of 80-100 kHz was applied during spectral acquisition. The cross-polarization contact time was set to 2 ms. Spectra were collected with a recycle delay of 18.5 seconds. The number of scans was adjusted to obtain an adequate signal to noise ratio. The ¹³C chemical shift scale was referenced using a ¹³C CPMAS experiment on an external standard of crystalline adamantane, setting its upfield resonance to 29.5 ppm (as determined from neat TMS).

¹⁹F ssNMR spectra were collected using a proton decoupled magic angle spinning (MAS) experiment. A phase modulated proton decoupling field of 80-100 kHz was applied during spectral acquisition. Spectra were collected with a recycle delay of 37.5 seconds. The number of scans was adjusted to obtain an adequate signal to noise ratio. The ¹⁹F chemical shift scale was referenced using a ¹⁹F MAS experiment on an external standard of trifluoroacetic acid (50%/50% v/v in H₂O), setting its resonance to −76.54 ppm.

Automatic peak picking was performed using Bruker-BioSpin TopSpin version 3.6 software. Generally, a threshold value of 5% relative intensity was used for preliminary peak selection. The output of the automated peak picking was visually checked to ensure validity and adjustments were manually made, if necessary. Although specific solid-state NMR peak values are reported herein, there does exist a range for these peak values due to differences in instruments, samples, and sample preparation. This is common practice in the art of solid-state NMR because of the variation inherent in peak positions. A typical variability for ¹³C and ¹⁹F chemical shift x-axis values is on the order of plus or minus 0.2 ppm for a crystalline solid. The solid-state NMR peak heights reported herein are relative intensities. Solid-state NMR intensities can vary depending on the actual setup of the experimental parameters and the thermal history of the sample.

### General Method 3

### Raman Spectroscopy

Instrument method: Raman spectra were collected using a Thermo Scientific iS50 FT-Raman accessory attached to the FT-IR bench. A CaF2 beam splitter was utilized in the FT-Raman configuration. The spectrometer was equipped with a 1064 nm diode laser and a room temperature InGaAs detector. Prior to data acquisition, instrument performance and calibration verifications were conducted using polystyrene. Samples were analyzed in glass NMR tubes, as tablets or in a suitable sample holder held static during data collection. The spectra were collected using 0.5 W of laser power and 512 co-added scans. The collection range was 3700-100 cm⁻¹. The API spectra were recorded using 2 cm⁻¹ resolution, and Happ-Genzel apodization was utilized for all of the spectra. Multiple spectra were recorded, and the reported spectrum is representative of two spots.

Peak picking method: The intensity scale was normalized to 1 prior to peak picking. Peaks were manually identified using the Thermo Nicolet Omnic 9.7.46 software. Peak position was picked at the peak maximum, and peaks were only identified as such, if there was a slope on each side; shoulders on peaks were not included. Peaks below 200 cm⁻¹ were not included in the peak table due to a rising background. An absolute threshold of 0.03 with a sensitivity of 75 was utilized during peak picking. The peak position has been rounded to the nearest whole number using standard practice (0.5 rounds up, 0.4 rounds down). Peaks with normalized peak intensity between (1-0.75), (0.74-0.30), (0.29-0) were labeled as strong, medium and weak, respectively.

Characteristic peak designation: The characteristic peaks for crystalline anhydrous Compound 1, Form 1 were chosen based on intensity, as well as peak position. Comparison to spectra generated on Formulation Placebo Blends were conducted to ensure the uniqueness of crystalline anhydrous Compound 1, Form 1.

### Example 1

### Synthesis and Characterization of N-(2-chloro-3-((5-chloro-3-methyl-4-oxo-3,4-dihdroquinazolin-6-yl)amino)-4-fluorophenyl)-3-fluoroazetidine-1-sulfonamide Free base

Compound 1 was prepared as described in Example 126, using Intermediates P5 and P9, of International Patent Application No. PCT/IB2021/054919. The methods used to prepare intermediates P5 and P9, as well as Example 126 of International Patent Application No. PCT/IB2021/054919, are reproduced below.

### Preparation of 6-Amino-5-chloro-3-methylquinazolin-4(3H)-one (Intermediate P5)

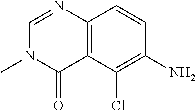

6-Amino-3-methylquinazolin-4(3H)-one (3.00 g, 17.1 mmol) was dissolved in THF (170 mL) and then treated with N-chlorosuccinimide (2.40 g, 18.0 mmol) and heated to 50° C. for 16 hours. The reaction mixture was treated with additional N-chlorosuccinimide (1.14 g, 8.56 mmol) and stirred at 50° C. for an additional 3 hours. The reaction mixture was concentrated, and the resulting residue was diluted with 1.0 M PXRD and extracted with DCM (3×). The combined DCM combined organic layers were washed with 1.0 M PXRD (2×) and the aqueous layer was neutralized with solid NaHCO₃ to about pH 7-8 and then extracted with 4:1 DCM:IPA (2×). The combined DCM:IPA extracts were dried over Na₂SO₄, filtered, and concentrated to provide 6-amino-5-chloro-3-methylquinazolin-4(3H)-one (2.47 g, 69%). ¹H NMR (400 MHz, DMSO) δ 8.10 (s, 1H), 7.38-7.36 (d, 2H), 7.29-7.26 (d, 2H), 5.81 (br-s, 2H), 3.40 (s, 3H). MS (apci, m/z)=210.1, 212.1 (M+H).

### Preparation of Tert-butyl (2-chloro-4-fluoro-3-iodophenyl)carbamate (Intermediate P9)

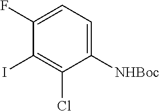 [SMILES: CC(C)(C)OC(=O)Nc1ccc(F)c(I)c1Cl]

Step 1: Preparation of 2-chloro-4-fluoro-3-iodoaniline. In a 5-L 4-neck flask equipped with 3 addition funnels, an internal temperature probe, and a magnetic stir bar, 2-chloro-4-fluoroaniline (82.03 mL, 687.0 mmol) was dissolved in THF (1.5 L) under a backflow of N₂ and cooled to −78° C.

The reaction mixture was treated dropwise with butyllithium (2.5 M in hexanes) (299.5 mL, 748.8 mmol) and allowed to stir at −78° C. for 15 minutes after complete addition. The reaction mixture was treated dropwise with a THF solution (500 mL) of 1,2-bis(chlorodimethylsilyl)ethane (155.3 g, 721.4 mmol) and allowed to stir at −78° C. for 30 minutes after complete addition. The reaction mixture was treated dropwise with additional butyllithium (2.5 M in hexanes) (299.5 mL, 748.8 mmol) and then the ice bath was removed after complete addition and the reaction mixture was stirred for 1 hour. The reaction mixture was cooled back to −78° C. and treated dropwise with additional butyllithium (2.5 M in hexanes) (299.5 mL, 748.8 mmol) and stirred at −78° C. for 30 minutes after complete addition. The reaction mixture was treated dropwise with a THF solution (600 mL) of iodine (249.3 g, 982.4 mmol) and the ice bath was removed, and reaction mixture allowed to warm to ambient temperature and stir for 16 hours. The reaction mixture was treated with 1000 mL water followed by hydrochloric acid (4.0 M aqueous solution) (601.1 mL, 2404.5 mmol) and allowed to stir at ambient temperature for 1 hr. The reaction mixture was neutralized to about pH 8 using solid NaHCO₃ and then treated with sodium thiosulfate (3.0 M aqueous solution) (801.5 mL, 2404.5 mmol) and allowed to stir at ambient temperature for 30 minutes. The reaction mixture was transferred to an extraction funnel, rinsing the flask with MTBE and water, and then the layers were separated. The organic layer was washed with brine (1×) and dried over Na₂SO₄, filtered, and concentrated to provide 2-chloro-4-fluoro-3-iodoaniline (186.49 g, 100%). ¹H NMR (400 MHz, DMSO) δ 6.97-6.93 (m, 1H), 6.81-6.77 (m, 1H), 5.41 (br-s, 2H).

Step 2: Preparation of bis-tert-butyl (2-chloro-4-fluoro-3-iodophenyl)carbamate. In a 3-L 1 neck flask, 2-chloro-4-fluoro-3-iodoaniline (186.49 g, 686.99 mmol) was dissolved in THF (2.0 L) and treated with 4-(dimethylamino)pyridine (8.39 g, 68.7 mmol) followed by addition of di-tert-butyl 52ecarbonate (314.87 g, 1442.7 mmol) and then stirred at ambient temperature for 1 hour open to air with a Vigreux column. The reaction mixture was concentrated to dryness. The resulting residue was dissolved in DCM (1 L) and diluted with hexanes (1 L) and stirred for 15 minutes, then passed through a small plug of silica eluting with additional 1:1 DCM:Hexanes (2.5 L). The filtrate was concentrated to dryness and the resulting solids were suspended in heptane (500 mL) and stirred at 80° C. for 30 minutes. The mixture was cooled to 0° C. in an ice bath and filtered, rinsed with additional chilled (0° C.) heptane (500 mL), and the light tan solids were collected to provide bis-tert-butyl (2-chloro-4-fluoro-3-iodophenyl)carbamate (145.5 g, 45%). ¹H NMR (400 MHz, DMSO) δ 7.55-7.51 (m, 1H), 7.32-7.28 (m, 1H), 1.33 (s, 18H).

Step 3: Preparation of tert-butyl (2-chloro-4-fluoro-3-iodophenyl)carbamate. Bis-tert-butyl (2-chloro-4-fluoro-3-iodophenyl)carbamate (331.7 g, 703.2 mmol) was dissolved in MeOH (1.8 L) and treated with potassium carbonate (106.9 g, 773.5 mmol) then heated to 65° C. for 1 hour. The reaction mixture was cooled to ambient temperature and poured into 6.0 L of water and stirred for 30 minutes. The mixture was filtered, rinsed with additional water (1000 mL), and collected the light tan solids to provide tert-butyl (2-chloro-4-fluoro-3-iodophenyl)carbamate (258.0 g, 99%). ¹H NMR (400 MHz, DMSO) δ 8.82 (s, 1H), 7.53-7.50 (m, 1H), 7.24-7.20 (m, 1H), 1.42 (s, 9H).

### Preparation of Tert-butyl (2-chloro-4-fluoro-3-iodophenyl)((3-fluoroazetidin-1-yl)sulfonyl)carbamate

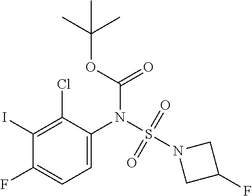 [SMILES: CC(C)(C)OC(=O)N(c1ccc(F)c(I)c1Cl)S(=O)(=O)N1CC(F)C1]

To a solution of tert-butyl (2-chloro-4-fluoro-3-iodophenyl)carbamate (Intermediate P9) (100 mg, 0.269 mmol) in tetrahydrofuran (1790 μL) at 0° C. was added sodium hydride (60% in mineral oil, 16 mg, 0.40 mmol) and stirred for 10 minutes. 3-Fluoroazetidine-1-sulfonyl chloride (70 mg, 0.40 mmol) was added and the solution was heated to 50° C. for 5 hours. The solution was then partitioned between dichloromethane and saturated NaHCO₃ and then the organic layer was washed with brine, dried over Na₂SO₄, filtered, concentrated, and purified by silica gel chromatography (5-95% EtOAc/hexanes) to give tert-butyl (2-chloro-4-fluoro-3-iodophenyl)((3-fluoroazetidin-1-yl)sulfonyl)carbamate (60 mg, 44% yield).

### Preparation of N-(2-chloro-3-((5-chloro-3-methyl-4-oxo-3,4-dihydroquinazolin-6-yl)amino)-4-fluorophenyl)-3-fluoroazetidine-1-sulfonamide

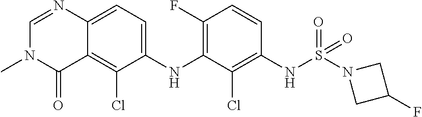 [SMILES: Cn1cnc2ccc(Nc3c(F)ccc(NS(=O)(=O)N4CC(F)C4)c3Cl)c(Cl)c2c1=O]

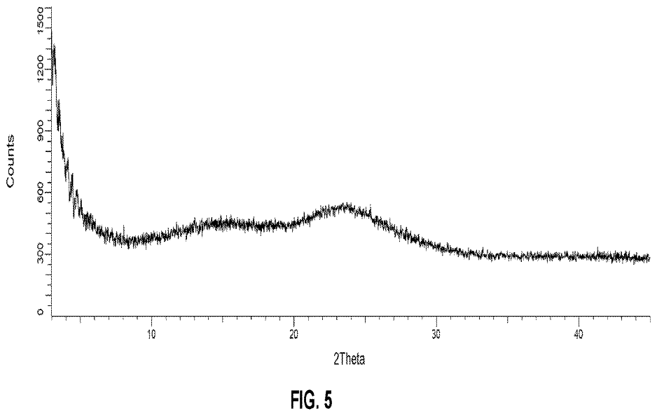

A solution of 6-amino-5-chloro-3-methylquinazolin-4(3H)-one (Intermediate P5; 90 mg, 0.42 mmol), tert-butyl (2-chloro-4-fluoro-3-iodophenyl)((3-fluoroazetidin-1-yl)sulfonyl)carbamate (218 mg, 0.429 mmol), tris(dibenzylideneacetone)dipalladium (39 mg, 0.042 mmol), Xantphos (62 mg, 0.10 mmol), and cesium carbonate (279 mg, 0.858 mmol) in toluene (2860 μL) was sparged with argon and heated to 110° C. overnight in a sealed vial. The solution was filtered through Celite®, concentrated, and the residue was stirred in 1 mL of DCM and 1 mL of TFA for 1 hour. The solution was concentrated and purified by reverse-phase chromatography (5-95% MeCN/water, 0.1% TFA) and the product was partitioned between DCM and saturated NaHCO₃. The organic layer was washed with brine, dried over Na₂SO₄, filtered, and concentrated to give N-(2-chloro-3-((5-chloro-3-methyl-4-oxo-3,4-dihydroquinazolin-6-yl)amino)-4-fluorophenyl)-3-fluoroazetidine-1-sulfonamide (78 mg, 37% yield). ¹H NMR (400 MHz, CDCl₃) δ 7.94 (s, 1H), 7.56-7.52 (m, 1H), 7.51 (d, 1H), 7.19-7.14 (t, 1H), 6.99-6.95 (m, 1H), 6.72 (s, br, 1H), 6.47 (s, br, 1H), 5.35-5.15 (m, 1H), 4.25-4.10 (m, 4H), 3.57 (s, 3H); MS (apci, m/z)=490.1, 492.1 (M+H). The material isolated from this step was analyzed by PXRD. The PXRD pattern, which is shown in FIG. 5, confirmed that the material is amorphous. On this basis the material was designated as amorphous Compound 1, Form 2.

### Example 2

### Alternative Preparation of amorphous N-(2-chloro-3-((5-chloro-3-methyl-4-oxo-3,4-dihydroquinazolin-6-yl)amino)-4-fluorophenyl)-3-fluoroazetidine-1-sulfonamide, Form 2

Crude N-(2-chloro-3-((5-chloro-3-methyl-4-oxo-3,4-dihydroquinazolin-6-yl)amino)-4-fluorophenyl)-3-fluoroazetidine-1-sulfonamide was taken up in 2-propanol (5 mL/g) and water (5 mL/g). To this mixture was added 1 N NaOH (1.0 eq) to increase the pH to >12. Upon full dissolution the solution was passed through a speck-free filter to provide a clear solution. The pH was rapidly adjusted to <4 by addition of 1N PXRD resulting in precipitation of the amorphous Compound 1, Form 2. The solids were isolated by filtration and washed with water.

### Example 3

### Alternative Preparation of N-(2-chloro-3-((5-chloro-3-methyl-4-oxo-3,4-dihydroquinazolin-6-yl)amino)-4-fluorophenyl)-3-fluoroazetidine-1-sulfonamide, amorphous Form 2

This example describes a method of preparing amorphous Compound 1 Form 2 using a melt quench method. N-(2-chloro-3-((5-chloro-3-methyl-4-oxo-3,4-dihydroquinazolin-6-yl)amino)-4-fluorophenyl)-3-fluoroazetidine-1-sulfonamide (prepared according to Example 1) was heated to 210° C. at a heating rate of 2° C./minute in-situ in a DSC instrument, then quench cooled at 60° C./minute to −20° C. and brought to ambient temperature to provide amorphous Compound 1, Form 2. The lack of a melting peak in the second heat and lack of recrystallization peak in the cool cycle were evidence that the material obtained when brought back up to ambient temperature was amorphous.

### Example 4

### Preparation and characterization of crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1

Crude N-(2-chloro-3-((5-chloro-3-methyl-4-oxo-3,4-dihydroquinazolin-6-yl)amino)-4-fluorophenyl)-3-fluoroazetidine-1-sulfonamide (prepared according to Example 1; 42.92 grams) was charged to a mixture of isopropanol (171 mL) and water (171 mL). Full dissolution was achieved by the addition of 1M aqueous sodium hydroxide (96 mL, 1.0 eq) to adjust the pH to 12.9. The solution was filtered through a speck-free filter (0.45 μm Nylon filter) and then warmed to 50° C. The desired material was crystallized by the slow addition of 1 M aqueous hydrochloric acid (84 mL, 1.0 eq) in a dropwise fashion over 2 hours and controlling the pH endpoint to pH=4 to 6. The resulting slurry was cooled from 50° C. to 20° C. at 0.3° C./min and then granulated at 20° C. for at least 2 hours. The solid was collected by filtration and washed with a 50% mixture of isopropanol and water (172 mL) followed by a water wash (172 ml). The isolated product was dried under vacuum with a nitrogen flush to afford 41.57 g of crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 (“crystalline anhydrous Compound 1, Form 1”).

A PXRD pattern of the crystalline anhydrous Compound 1, Form 1 is shown in FIG. 1. A full PXRD peak list and relative intensity data for the crystalline anhydrous Compound 1, Form 1 is provided in Table 1. Characterizing PXRD peaks were identified as peaks at 8.3, 11.5 and 16.1 degrees 2-theta (±0.2 degrees 2-theta). The ¹⁹F solid state NMR spectrum of the crystalline anhydrous Compound 1, Form 1 is shown in FIG. 2. ¹⁹F solid state NMR peaks are provided in Table 2. The ¹³C solid state NMR of the crystalline anhydrous Compound 1, Form 1 is shown in FIG. 3. The full list of ¹³C solid state NMR peaks is shown in Table 3A, and the characteristic peaks are shown in Table 3B. The Raman spectrum of the crystalline anhydrous Compound 1, Form 1 is shown in FIG. 4. The full Raman peak list for the crystalline anhydrous Compound 1, Form 1 is shown in Table 4A, and characteristic peaks are listed in Table 4B.

### Example 5

### Physical Stability Analysis

The physical stabilities of crystalline anhydrous Compound 1, Form 1, and amorphous Compound 1, Form 2 were evaluated using PXRD on samples stored at either 5° C. or at 70° C./75% relative humidity (RH) for 8 days. Table 5 details the differences between Form 1 and amorphous form.

TABLE 5

| | | Solid form identified |
|---|---|---|
| Starting material | Condition | by PXRD after 8 days |
| amorphous Compound | 70° C./75% RH | crystalline anhydrous |
| 1, Form 2 | | Compound 1, Form 1 |
| amorphous Compound | 5° C. | amorphous compound 1, |
| 1, Form 2 | | Form 2 |
| crystalline anhydrous | 70° C./75% RH | crystalline anhydrous |
| Compound 1, Form 1 | | Compound 1, Form 1 |
| crystalline anhydrous | 5° C. | crystalline anhydrous |
| Compound 1, Form 1 | | Compound 1, Form 1 |

The data demonstrate that under the accelerated stability conditions tested on storage at 70° C./75% relative humidity (RH) for 8 days, crystalline anhydrous Compound 1, Form 1 was stable under these conditions, but amorphous Compound 1, Form 2 crystallized to crystalline anhydrous Compound 1, Form 1.

### Example 7

### Hygroscopicity Data

Water sorption and desorption were analyzed with a DVS-Resolution. The microbalance was calibrated with a 100 mg standard weight on a monthly basis. About 8 mg of the compound was added to a sample pan and placed inside chamber A of the DVS-Resolution. Percent relative humidity was held at 0% for 1 hour, and increased to 90% in 10% increments, then ramped back to 0% in 10% increments. Steps were considered complete when a dm/dt change of <0.001% was observed with a minimum of 30 minutes or a maximum step time of 120 minutes was reached. The powder material remaining following the DVS analysis was analyzed by PXRD to determine if a change in the diffraction pattern is observed. The data are shown in Table 6. Based on this analysis, the crystalline anhydrous Compound 1, Form 1 is not hygroscopic, and amorphous Compound 1, Form 2 is hygroscopic with approximately 3.6% weight gain at 25° C. and 90% RH.

| | Mass Gain at 25° | Mass Gain at 25° | | |
|---|---|---|---|---|
| Starting material | C. and 60% RH | C. and 90% RH | Hygroscopicity | PXRD Post-DVS |
| amorphous compound | 1.7 | 3.6 | Hygroscopic | amorphous Compound |
| 1, Form 2 | | | | 1, Form 2 |
| crystalline anhydrous | 0.0 | 0.0 | Non-Hygroscopic | crystalline anhydrous |
| Compound 1, Form 1 | | | | Compound 1, Form 1 |

### BIOLOGICAL EXAMPLES

### Example A1

### BRAF V600E Enzyme Assay

A competitive displacement assay was configured for B-Raf that monitors the amount of a fluorescently-tagged “tracer” bound to B-Raf via TR-FRET from an anti-tag Eu-labeled antibody also bound to B-Raf. For full-length FLAG-tagged B-Raf(V600E), the assay mixtures consisted of 25 mM K⁺HEPES, pH 7.4, 10 mM MgCl₂, 0.01% Triton X-100, 1 mM DTT, 2% DMSO (from compound), 50 nM Tracer 1710 (ThermoFisher, PR9176A), 0.5 nM Eu anti-FLAG (M2)-cryptate Ab (Cisbio, 61FG2KLB) and 5 nM full-length, N-terminally FLAG-tagged B-Raf(V600E) (Origene Technologies, TP700031. N-(2-chloro-3-((5-chloro-3-methyl-4-oxo-3,4-dihydroquinazolin-6-yl)amino)-4-fluorophenyl)-3-fluoroazetidine-1-sulfonamide was diluted in DMSO across an 11-point dosing range created using a 3-fold serial dilution protocol at a top dose of 10 μM. The assay was run in 384-well, polystyrene, low-volume, non-treated, white microtiter plates (Costar 4512) in a final volume of 12 μL. Low control wells included 1 μM of a potent B-Raf inhibitor as a control. The assays were incubated at ambient temperature (typically 22° C.) for 60 min and then read on a PerkinElmer EnVision microplate reader using standard TRF settings (λ_{Ex}=320 nm, λ_{Em}=615 & 665 nm). The ratioed counts (665 nm/615 nm) were converted to percent of control (POC) using the following equation:

$$POC = \frac{Sample - {\overline{X}}_{min}}{{\overline{X}}_{max} - {\overline{X}}_{min}} \times 100$$

where:
   x̅ₘₐₓ Average Uninhibited Controls
   X̅ₘᵢₙ Average Background

A 4-parameter logistic model was the fit to the POC data for each compound. From that fit, the IC₅₀ was estimated and is defined as the concentration of compound at which the best-fit curve crosses 50 POC and is provided in Table A.

### Example A2

### Cellular phospho-ERK Inhibition Assays in A375 and H1755 Cells

This Example describes two cellular assays used to test the ability of N-(2-chloro-3-((5-chloro-3-methyl-4-oxo-3,4-dihydroquinazolin-6-yl)amino)-4-fluorophenyl)-3-fluoroazetidine-1-sulfonamide (Compound 1) to inhibit phosphor-ERK.

A375 and H1755 cells were obtained from the American Type Culture Collection (ATCC, Rockville, MD). A375 cells were maintained in DMEM growth medium containing 10% FBS. H1755 cells were maintained in RPMI growth medium containing 10% FBS.

Cells were harvested according to standard protocols, counted and plated onto flat-bottom, 96-well tissue culture plates (Costar No. 3599) at 2.5×10⁴ cells/well for A375 cells and 1.5×10⁴ cells/well for H1755 cells in 100 μL/well of growth medium containing 10% FBS. After an overnight incubation at 37° C. with 5% CO₂ cells were treated for 2 hours at 37° C., 5% CO₂ with compounds prepared as a 9-point, 1:3.33 fold dilution series with final compound concentrations ranging from 66 pM-10 μM and a constant DMSO concentration of 0.25%. Control wells contained either 0.25% DMSO alone (uninhibited control) or 10 μM binimetinib (complete inhibition control). The levels of phosphorylated ERK are determined using an In Cell Western protocol: following compound incubation, growth medium was discarded, and cells were fixed with 0.4% formaldehyde in PBS for 20 minutes at room temperature. Cells were permeabilized with 100% methanol for 10 minutes at room temperature. Plates were washed with PBS containing 0.05% Tween-20 and blocked for 1 hour at room temperature with LI-COR Blocking Buffer (LI-COR Biosciences; Cat #927-40000). Plates were then incubated for 2 hours at room temperature with 50 μL of a 1:400 dilution of anti-phospho-ERK1/2 (Thr202/Tyr204) (Cell Signaling; Catalog No. 9101) and a 1:1000 dilution of anti-GAPDH (Millipore; Catalog No. MAB374) in LI-COR blocking buffer containing 0.05% Tween-20. Plates were washed with PBS containing 0.05% Tween-20 then incubated at room temperature for 1 hour with 50 μL of a 1:1000 dilution of anti-rabbit AlexaFluor 680 (Life Technologies; Catalog No. A21109) and a 1:1000 dilution of anti-mouse IRDye 800CW (LI-COR; Catalog No. 926-32210) in LI-COR blocking buffer containing 0.05% Tween-20. Plates were analyzed by reading on an Odyssey CLx infrared scanner. For each well, the phospho-ERK signal was normalized to the GAPDH signal and converted to POC using the following equation:

$$POC = \frac{Sample - {\overline{X}}_{min}}{{\overline{X}}_{max} - {\overline{X}}_{min}} \times 100$$

| where: | |
|---|---|
| x̅ₘₐₓ | Average Uninhibited Controls |
| X̅ₘᵢₙ | Average Complete Inhibition Controls |

The IC₅₀ values were then calculated using a 4-parameter fit in XLfit software and are provided in Table A1.

TABLE A

| BRAF V600E enzyme IC₅₀ | A357 cell IC₅₀ | H1755 Cell IC₅₀ |
|---|---|---|
| (nM) | (nM) | (nM) |
| 3 | 2 | 26 |

### Example A3

### Cellular Phospho-ERK Inhibition Assay

N-(2-chloro-3-((5-chloro-3-methyl-4-oxo-3,4-dihydroquinazolin-6-yl)amino)-4-fluorophenyl)-3-fluoroazetidine-1-sulfonamide (Compound 1) was evaluated in a phospho-ERK assay in two mutant BRAF Class Ill cell lines: NCI-H1666 (BRAF^{G466V}) and WM3629 cells (BRAF^{D594G}/NRAS^{G12D}) NCI-H1666 cells were obtained from the American Type Culture Collection (ATCC, Rockville, MD) and WM3629 cells were obtained from Rockland Immunochemicals (Limerick, PA). Cells were maintained in RPMI growth medium containing 10% FBS.

Cells were harvested according to standard protocols, counted, and plated onto flat-bottom, 96-well tissue culture plates (Costar No. 3599) at 2.5×10⁴ cells/well in 100 μL/well of growth medium containing 10% FBS. After an overnight incubation at 37° C. with 5% CO₂ cells were treated for 1 hour at 37° C., 5% CO₂ with inhibitors prepared as a 9-point, 1:3.33 fold dilution series with final compound concentrations ranging from 66 pM-10 μM and a constant DMSO concentration of 0.25%. Control wells contained either 0.25% DMSO alone (uninhibited control) or 10 μM binimetinib (complete inhibition control). The levels of phosphorylated ERK were determined using an In Cell Western protocol: following compound incubation, growth medium was discarded, and cells were fixed with 0.4% formaldehyde in PBS for 20 minutes at room temperature. Cells were permeabilized with 100% methanol for 10 minutes at room temperature. Plates were washed with PBS containing 0.05% Tween-20 and blocked for 1 hour at room temperature with LI-COR Blocking Buffer (LI-COR Biosciences; Cat. #927-40000). Plates were then incubated for 2 hours at room temperature with 50 μL of a 1:400 dilution of anti-phospho-ERK1/2 (Thr202/Tyr204) (Cell Signaling; Cat. #9101) and a 1:1000 dilution of anti-GAPDH (Millipore; Cat. #MAB374) in LI-COR blocking buffer containing 0.05% Tween-20. Plates were washed with PBS containing 0.05% Tween-20 then incubated at room temperature for 1 hour with 50 μL of a 1:1000 dilution of anti-rabbit AlexaFluor 680 (Life Technologies; Catalog No. A21109) and a 1:1000 dilution of anti-mouse IRDye 8000W (LI-COR; Catalog No. 926-32210) in LI-COR blocking buffer containing 0.05% Tween-20. Plates were analyzed by reading on an Odyssey CLx infrared scanner. For each well, the phospho-ERK signal was normalized to the GAPDH signal and converted to POC using the following equation:

$$POC = \frac{Sample - {\overline{X}}_{min}}{{\overline{X}}_{max} - {\overline{X}}_{min}} \times 100$$

Where:
   X̅ₘₐₓ d Average Uninhibited Controls
   X̅ₘᵢₙ Average Complete Inhibition Controls

IC₅₀ values were then calculated using a 4-parameter fit in XLfit software and are shown in Table A2.

### Example A4

### Proliferation Assay

N-(2-chloro-3-((5-chloro-3-methyl-4-oxo-3,4-dihydroquinazolin-6-yl)amino)-4-fluorophenyl)-3-fluoroazetidine-1-sulfonamide (Compound 1) was evaluated in a proliferation assay in two mutant BRAF Class III cell lines: NCI-H1666 (BRAF^{G466V}) and WM3629 cells (BRAF^{D594G}/NRAS^{G12D}). NCI-H1666 cells were obtained from the American Type Culture Collection (ATCC, Rockville, MD) and WM3629 cells were obtained from Rockland Immunochemicals (Limerick, PA). Cells were maintained in RPMI growth medium containing 10% FBS.

Cells were harvested according to standard protocols, counted, and plated onto flat-bottom, 96-well tissue culture plates (Costar #3599) at 2000-5000 cells/well in 100 μL/well of growth medium containing 10% FBS. Cells were incubated at 37° C. with 5% CO₂ overnight then treated with inhibitors prepared as a 9-point, 1:3.33 fold dilution series with final compound concentrations ranging from 66 pM-10 μM and a constant DMSO concentration of 0.25%. Control wells contained 0.25% DMSO alone. After a 3-5 day incubation at 37° C., 5% CO₂, cell viability was determined by adding 100 μL CellTiter-Glo© Reagent (Promega) to each well and incubated for 15 minutes at room temperature. “Day 0” controls were determined by performing CellTiter-Glo® assay on DMSO control wells at the time of compound treatment (“Day 0” control=0 POC). Luminescence was measured on a Cytation 5 plate reader (BioTek) and values were converted to POC using the following equation:

$$POC = \frac{Sample - {\overline{X}}_{min}}{{\overline{X}}_{max} - {\overline{X}}_{min}} \times 100$$

Where:
   X̅ₘₐₓ Average DMSO Controls
   X̅ₘᵢₙ Average “Day 0” DMSO Controls

IC₅₀ values were calculated using a 4-parameter fit in XLfit software and are shown in Table A2.

TABLE A2

| | |
|---|---|
| NCI-H1666 | WM3629 |

| pERK | Proliferation | pERK | Proliferation |
|---|---|---|---|
| IC₅₀ (nM) | IC₅₀ (nM) | IC₅₀ (nM) | IC₅₀ (nM) |
| 15 | 390 | 1.4 | >10000 |

### Example B

### MDR1 LLC-PK1 and BCRP MDCKII Permeability Assay

Both LLC-PK1 and MDR1 transfected LLC-PK1 cells were cultured and plated according to manufacturer's recommendations with the exception that the passage media contained only 2% fetal bovine serum to extend passage time out to seven days.

BCRP transfected MDCKII cells were cultured and plated according to manufacturer's recommendations. Assay conditions included with and without the BCRP-specific inhibitor, K0143, at a concentration of 0.3 μM to ascertain the contribution of BCRP to the efflux value of the test compound.

Both positive and negative controls were used to assess functionality of P-gp or BCRP efflux in the assays. Stock solutions for assay controls and the test article were prepared in DMSO for final test concentrations of 10 and 1 μM, respectively. Final organic concentration in the assay was 1%. All dosing solutions contained 10 μM lucifer yellow to monitor LLC-PK1 or MDCKII cell monolayer integrity.

For the apical to basolateral determination (A to B), 75 μL of the test article, N-(2-chloro-3-((5-chloro-3-methyl-4-oxo-3,4-dihydroquinazolin-6-yl)amino)-4-fluorophenyl)-3-fluoroazetidine-1-sulfonamide (Compound 1), in transport buffer was added to the apical side of the individual transwells and 250 μL of basolateral media, without compound or lucifer yellow, were added to each well. For the basolateral to apical determination (B to A), 250 μL of test article in transport buffer were added to each well and 75 μL transport buffer, without compound or lucifer yellow, were added to each transwell. All tests were performed in triplicate, and the compound was tested for both apical to basolateral and basolateral to apical transport. The plates were incubated for 2 hours on a Lab-Line Instruments Titer Orbital Shaker (VWR, West Chester, PA) at 50 rpm and 37° C. with 5% CO₂. All culture plates were removed from the incubator and 50 μL of media were removed from the apical and basolateral portion of each well and added to 150 μL of 1 μM labetalol in 2:1 acetonitrile (acetonitrile): H₂O, v/v.

The plates were read using a Molecular Devices (Sunnyvale, CA) Gemini Fluorometer to evaluate the lucifer yellow concentrations at excitation/emission wavelengths of 425/535 nm. These values were accepted when found to be below 2% for apical to basolateral and 5% basolateral to apical flux across the MDR1-transfected LLC-PK1 or BCRP-transfected MDCKII cell monolayers. The plates were sealed and the contents of each well analyzed by LC-MS/MS. The compound concentrations were determined from the ratio of the peak areas of the compound to the internal standard (labetalol) in comparison to the dosing solution.

### LC-MS Analysis

The LC-MS/MS system was comprised of an HTS-PAL autosampler (Leap Technologies, Carrboro, NC), an HP1200 HPLC (Agilent, Palo Alto, CA), and a MDS Sciex 4000 Q Trap system (Applied Biosystems, Foster City, CA). Chromatographic separation of the analyte and internal standard was achieved at room temperature using a C18 column (Kinetics®, 50×300 mm, 2.6 μm particle size, Phenomenex, Torrance, CA) in conjunction with gradient conditions using mobile phases A (water containing 1% isopropyl alcohol and 0.1% formic acid) and B (0.1% formic acid in acetonitrile). The total run time, including re-equilibration, for a single injection was 1.2 minutes. Mass spectrometric detection of the analytes was accomplished using the ion spray positive mode. Analyte responses were measured by multiple reaction monitoring (MRM) of transitions unique to each compound (the protonated precursor ion and selected product ions for each test article and m/z 329 to m/z 162 for labetalol, the internal standard).

The permeability coefficient (Pₐₚₚ) is calculated from the following equation:

$${P}_{app} = [ ( ( {C}_{d} * V * ( 1 \times {10}^{6} ) ) / ( t * 0.12 {cm}^{2} * C ) ]$$

where C_{d}, V, t and C₀ are the detected concentration (μM), the volume on the dosing side (mL), the incubation time (s) and the initial dosing concentration (μM), respectively. The calculations for Pₐₚₚ were made for each replicate and then averaged. The permeability coefficient for Compound 1 when tested in this assay was 44*10⁻⁶ cm/s. In this assay, a compound is defined has having high permeability if the permeability is greater than 8×10⁻⁶ cm/sec.

An efflux ratio was calculated from the mean apical to basolateral (A-B) Pₐₚₚ data and basolateral to apical (B-A) Papp data using the following equation:

$$Efflux ratio = {P}_{app} ( B - A ) / {P}_{app} ( A - B )$$

The efflux ratios for N-(2-chloro-3-((5-chloro-3-methyl-4-oxo-3,4-dihydroquinazolin-6-yl)amino)-4-fluorophenyl)-3-fluoroazetidine-1-sulfonamide (Compound 1) when tested in this assay were 2.1 (MDR1) and 4.2 (BCRP).

### Example C

### PK (Free Brain-to-Free Plasma Ratio) (Mouse)

The ability of N-(2-chloro-3-((5-chloro-3-methyl-4-oxo-3,4-dihydroquinazolin-6-yl)amino)-4-fluorophenyl)-3-fluoroazetidine-1-sulfonamide (Compound 1) to penetrate the BBB in mice was determined by evaluating the unbound brain-to-unbound plasma (also referred to as free brain-to-free plasma) concentration ratio in male CD-1 mice.

Brain compound levels were generated from oral mouse PK dosing with typical sampling times of 2, 4, 8, 12 and 24 hours post oral gavage dosing at 10 mg/kg. Brain samples were stored at −20±5° C. prior to analysis. Concentrations of Compound 1 in mouse brain homogenate were determined by liquid chromatography tandem mass spectrometry (LC-MS/MS) following protein precipitation with acetonitrile. A 12-point calibration curve, ranging from 0.5 to 10,000 ng/mL, was prepared in duplicate. A solution of 400 μg/mL of Compound 1 in dimethyl sulfoxide (DMSO) was serially diluted (3-fold) in 100% DMSO, and then 2.5 μL of each standard solution was added to 100 μL of naïve male CD-1 mice brain homogenate. To mimic the extraction in the standard curve, 2.5 μL of DMSO was added to all test samples. Both calibration and test brain homogenate samples were spiked with 10 μL of an IS (1 μg/mL of a structural analog). Brain homogenate was generated by adding 0.75 mL of 4:1 water:MeOH to each brain sample followed by homogenization for 1 minute with bead beater tubes a 6 m/s using an MP Fast Prep-24®. Proteins were precipitated from 100 μL of brain homogenate sample by the addition of 300 μL of acetonitrile. Samples were vortex-mixed for 5 minutes and spun in an Allegra X-12R centrifuge (Beckman Coulter, Fullerton, CA; SX4750A rotor) for 15 min at approximately 1,500×g at 4° C. A 100 μL aliquot of each supernatant was transferred via a 550 μL Personal Pipettor (Apricot Designs, Monrovia, CA) to 96-well plates and diluted 1:1 with HPLC grade water. The resulting plates were sealed with aluminum for LC-MS/MS analysis.

Brain-to-plasma ratios were calculated using the concentration of Compound 1 measured in the brain divided by the concentration of compound measured in the plasma. Brain-to-plasma ratios were always generated from a single animal and time point. Free brain-to-free plasma ratios were calculated by multiplying the brain-to-plasma ratio by the in vitro brain homogenate free fraction divided by the in vitro plasma free fraction using the following equation: (B/P)*(B_{fu}/P_{fu}). The free brain-to-free plasma ratio of Compound 1 when tested in this assay was 0.12-0.17.

Variations, modifications, and other implementations of what is described herein will occur to those skilled in the art without departing from the spirit and the essential characteristics of the present teachings. Accordingly, the scope of the present teachings is to be defined not by the preceding illustrative description but instead by the following claims, and all changes that come within the meaning and range of equivalency of the claims are intended to be embraced therein.

Each of the printed publications, including but not limited to patents, patent applications, books, technical papers, trade publications and journal articles described or referenced in this specification are herein incorporated by reference in their entirety and for all purposes.

I claim:

1. A crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 having a ¹⁹F solid state NMR spectrum comprising resonance values of −188.1 and −115.8 ppm±0.2 ppm.

2. The crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to claim 1, having a ¹³C solid state NMR spectrum comprising resonance (ppm) values at 35.8, 57.5, 130.6 and 148.1 ppm±0.2 ppm.

3. The crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to claim 1 or 2, having a powder X-ray diffraction pattern measured using copper wavelength radiation comprising peaks, in terms of 2-theta, at 8.3, 11.5 and 16.1 degrees 2-theta±0.2 degrees 2-theta.

4. The crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to any one of claims 1 to 3, having a powder X-ray diffraction pattern measured using copper wavelength radiation comprising peaks, in terms of 2-theta, at 8.3, 11.5, 16.1, 22.9, and 23.6 degrees 2-theta (±0.2 degrees 2-theta)

5. The crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to any one of claims 1 to 4, having a Raman spectrum comprising one or more wavenumber (cm⁻¹) values at 1308, 1433, 1447, 1548 and 1608 cm⁻¹±2 cm⁻¹.

6. A pharmaceutical composition comprising the crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to any one of claims 1 to 5, and one or more pharmaceutically acceptable excipients.

7. The pharmaceutical composition according to claim 6, further comprises microcrystalline cellulose, dibasic calcium phosphate anhydrous, crospovidone Type B and sodium stearyl fumarate.

8. A method of treating a BRAF-associated tumor in a subject in need thereof, comprising administering to the subject the crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to any one of claims 1 to 5.

9. The method according to claim 8, wherein said BRAF-associated tumor has a BRAF Class II mutation.

10. The method according to claim 8 or 9, wherein said BRAF-associated tumor is a cancer selected from lung cancer, melanoma, colorectal cancer, breast cancer, pancreatic cancer, thyroid cancer, prostate cancer, adenoid cystic carcinoma, appendiceal cancer, small intestine cancer, head and neck squamous cell carcinoma, angiosarcoma, bladder carcinoma, plasma cell neoplasm, hepato-pancreato-biliary carcinoma, ovary carcinoma, neuroendocrine cancer, cholangiocarcinoma and CNS cancers.

11. The method according to claim 8, wherein said BRAF-associated tumor has a BRAF Class I mutation.

12. The method according to claim 11, wherein said BRAF-associated tumor is selected from melanoma, colorectal cancer, thyroid cancer, non-small cell lung cancer, ovarian cancer, renal cell carcinoma, and metastatic cancers thereof, and primary brain tumors.

13. The method according to any one of claims 8 to 12, wherein the method further comprises administering one or more additional anticancer agents.

14. The method according to claim 13, wherein the additional anticancer agent is a MEK inhibitor.

15. The method of claim 14, wherein the MEK inhibitor is binimetinib or a pharmaceutically acceptable salt thereof.

16. The method of claim 13, wherein the additional anticancer agent is an EGFR inhibitor, wherein the EGFR inhibitor is cetuximab.

17. The method of claim 13, wherein the additional anticancer agents are a MEK inhibitor and an EGFR inhibitor, wherein the MEK inhibitor is binimetinib or a pharmaceutically acceptable salt thereof, and the EGFR inhibitor is cetuximab.

18. The crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to any one of claims 1 to 5 for use as a medicament.

19. The crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to any one of claims 1 to 5 for use in the treatment of a BRAF-associated tumor.

20. Use of the crystalline anhydrous N-(2-chloro-3-((5-chloro-3-methyl-4-oxo-3,4-dihydroquinazolin-6-yl)amino)-4-fluorophenyl)-3-fluoroazetidine-1-sulfonamide, Form 1 according to any one of claims 1 to 5 for the manufacture of a medicament for the treatment of a BRAF-associated tumor.
